(19) United States
(12) Patent Application Publication
(10) Pub. No.: US 2026/0265385 A1
(43) Pub. Date: Sep. 10, 2026

(54) CD122 BINDING AGENTS AND METHOD OF USING SAME

(71) Applicant: AnaptysBio, Inc., San Diego, CA (US)

(72) Inventors: Martin Edward Dahl, San Diego, CA (US); Stephen Parmley, San Diego, CA (US); Eric Hare, San Diego, CA (US); Chris Haines, San Diego, CA (US); Robert Morse, San Diego, CA (US)

(21) Appl. No.: 19/126,534

(22) PCT Filed: Nov. 7, 2023
(86) PCT No.: PCT/US2023/078989

§ 371 Date: May 1, 2025

Related U.S. Application Data
(60) Provisional application No. 63/423,416, filed on Nov. 7, 2022.

(51) Int. Cl. C07K 16/28

(52) U.S. Cl. CPC C07K 16/2866; C07K 2317/24; C07K 2317/622

(57) ABSTRACT

Provided is a CD122 binding agent comprising an immunoglobulin heavy chain polypeptide and immunoglobulin light chain polypeptide, as well as related compositions and methods for making and using same.

### CROSS-REFERENCE TO RELATED APPLICATIONS

This application claims priority to U.S. provisional patent application No. 63/423,416 filed Nov. 7, 2022, the entire disclosure of which is hereby incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 214,653 Byte ASCII (Text) file named “769570_ST.26.xml,” created on Nov. 2, 2023.

### BACKGROUND OF THE INVENTION

Interleukin-2 (IL-2) and Interleukin-15 (IL-15) are two important cytokines involved in immune system regulation. IL-2 is involved in regulatory T-cell (Treg) cell development and homeostasis, whereas IL-15 regulates NK and NK_T-cell development and memory T-cell survival. The receptors for IL-2 (IL-2R) and IL-15 (IL-15R) are each comprised of three subunits designated a, p, and 7. While IL-2R and IL-15R have unique a subunits (CD25 and CD125, respectively), the two receptors share common β and γ subunits (CD122 and CD132).

IL-2 and IL-15 signaling is implicated in pathogenic immune system responses. Thus, there is a need for CD122 binding agents that can modulate such signaling.

### BRIEF SUMMARY OF THE INVENTION

Provided herein is a CD122 binding agent comprising an immunoglobulin heavy chain and an immunoglobulin light chain, wherein, the immunoglobulin heavy chain variable region comprises SEQ ID NO: 113, or at least the CDR regions thereof, and the immunoglobulin light chain variable region comprises SEQ ID NO: 114, or at least the CDR regions thereof.

In another aspect, the disclosure provides a CD122 binding agent comprising an immunoglobulin heavy chain and an immunoglobulin light chain, wherein, the immunoglobulin heavy chain variable region comprises SEQ ID NO: 69, or at least the CDR regions thereof, and the immunoglobulin light chain variable region comprises SEQ ID NO: 77, or at least the CDR regions thereof.

In another aspect, the disclosure provides a CD122 binding agent comprising an immunoglobulin heavy chain and an immunoglobulin light chain, wherein, the immunoglobulin heavy chain variable region comprises SEQ ID NO: 150, or at least the CDR regions thereof, and the immunoglobulin light chain variable region comprises SEQ ID NO: 151, or at least the CDR regions thereof.

In another aspect, the disclosure provides a CD122 binding agent comprising an immunoglobulin heavy chain and an immunoglobulin light chain, wherein, the immunoglobulin heavy chain variable region comprises SEQ ID NO: 24, or at least the CDR regions thereof, and the immunoglobulin light chain variable region comprises SEQ ID NO: 35, or at least the CDR regions thereof.

The disclosure further provides a method for inhibiting an immune response and treating inflammatory or autoimmune disorders in a mammal by administering the CD122 binding agent to the mammal. Nucleic acid sequences encoding the foregoing immunoglobulin polypeptides and binding agents, and related compositions and methods also are provided.

### BRIEF DESCRIPTION OF THE SEVERAL VIEWS OF THE DRAWING(S)

FIG. 1 shows the results of a cell-based receptor binding assay illustrating ability of anti-CD122 antibodies to bind to CHO-K1 cells expressing human IL-2βγ.

FIG. 2 shows the results of a cell-based receptor binding assay illustrating ability of anti-CD122 antibodies to bind to CHO-K1 cells expressing cyno IL-2βγ.

FIG. 3 shows the results of a cell-based functional assay illustrating the ability of anti-CD122 antibodies to inhibit IL-2-induced pSTAT5 luciferase activity in HEK cells.

FIG. 4 shows the results of a CTG (cell titer glo)-based proliferation assay which illustrates the ability of humanized anti-CD122 IgG4 antibodies to inhibit IL-2 or IL-15-induced proliferation of PHA-stimulated primary human cell blasts.

FIG. 5 shows the binding activities of anti-CD122 IgG4 antibodies to CHO-K1 cells expressing human or cynomolgus IL-2βγ.

FIG. 6 shows the effects of anti-CD122 IgG4 antibodies on IL-2- or IL-15-induced proliferation of purified primary human NK cells.

FIG. 7 shows the effects of humanized anti-CD122 IgG4 antibodies on IL-2- or IL-15-induced proliferation and survival of primary human NK cells, gated within a PBMC culture.

FIG. 8 shows the effects of humanized anti-CD22 IgG4 antibodies on IL-2- or IL-15-binding to HEK cells expressing human IL-2βγ.

FIG. 9 shows the effects of humanized anti-CD122 IgG4 antibodies on IL-15 or IL-15/IL1-5RA induced proliferation of primary human T cell blasts.

FIG. 10 shows the effects of humanized anti-CD122 IgG4 antibodies on IL-15 or IL-15/IL1-5RA induced proliferation of primary human NK cells.

FIG. 11 shows the effects of humanized anti-CD122 IgG1 LALA antibodies on IL-2 or IL-15 induced proliferation of primary human NK cells.

FIG. 12 shows the effects of humanized anti-CD122 IgG1 LALA antibodies on IL-2- or IL-15-induced proliferation of primary human pan T cells.

FIG. 13 shows effects of humanized anti-CD122 antibodies in xenogeneic NSG (huIL-15)/Hu-PBMC GvHD model alone or in combination with CTLA4-Ig.

FIG. 14 shows effects of humanized anti-CD122 antibodies in xenogeneic NSG (huIL-15Tg)/Hu-PBMC Graft versus Host Disease (GvHD) model.

### DETAILED DESCRIPTION OF THE INVENTION

The invention provides a CD122 binding agent. The CD122 binding agent comprises an immunoglobulin heavy chain variable region and an immunoglobulin light chain variable region, each of which comprise three complementarity determining regions (CDRs), usually referred to as CDR1, CDR2, or CDR3. The CDR regions also can be referred to using an “H” or “L” in the nomenclature to denote the heavy or light chain, respectively, i.e., CDRH1, CDRH2, CDRH3, CDRL1, CDRL2, or CDRL3. The CDRs of a given Ig sequence can be determined by any of several conventional numbering schemes, such as Kabat, Chothia, Martin (Enhanced Chothia), IGMT, or AHo (see, e.g., Kabat, et al., Sequences of Proteins of Immunological Interest, U.S. Department of Health and Human Services, NIH (1991); Chothia, et al., Canonical Structures for the Hypervariable Regions of Immunoglobulins, J. Mol. Biol., 196:901-917 (1987); Al-Lazikani et al., Standard Conformations for the Canonical Structures of Immunoglobulins, J. Mol. Biol., 273:927- 948 (1997); Abhinandan et al., Analysis and Improvements to Kabat and Structurally Correct Numbering of Antibody Variable Domains, Mol. Immunol., 45: 3832-3839 (2008); Lefranc et al., The IMGT unique numbering for immunoglobulins, T cell Receptors and Ig-like domains, The Immunologist, 7: 132-136 (1999); Lefranc et al., IMGT unique numbering for immunoglobulin and T cell receptor variable domains and I superfamily V-like domains, Dev. Comp. Immunol., 27: 55-77 (2003); and Honegger et al., Yet another numbering scheme for immunoglobulin variable domains: an automatic modeling and analysis tool, J. Mol. Biol. 309: 657-670 (2001).

Provided herein is a CD122 binding agent comprising an immunoglobulin heavy chain variable region and an immunoglobulin light chain variable region, wherein the immunoglobulin heavy chain variable region comprises SEQ ID NO: 113, or at least the CDR regions thereof, and the immunoglobulin light chain variable region comprises SEQ ID NO: 114, or at least the CDR regions thereof, wherein the CDR regions are as determined by Kabat, Chothia, Martin (Enhanced Chothia), IGMT, or AHo.

Also provided herein is a CD122 binding agent comprising an immunoglobulin heavy chain variable region and an immunoglobulin light chain variable region, wherein the immunoglobulin heavy chain variable region comprises one of SEQ ID NO: 37-69, or at least the CDRs thereof; and the immunoglobulin light chain variable region comprises the CDRs of any of SEQ ID NOs: 70-77, wherein the CDRs are as determined in accordance with any of the various known immunoglobulin numbering schemes (e.g., Kabat, Chothia, Martin (Enhanced Chothia), IGMT, or AHo).

Also provided herein is a CD122 binding agent comprising an immunoglobulin heavy chain variable region and an immunoglobulin light chain variable region, wherein the immunoglobulin heavy chain variable region comprises an amino acid sequence with at least 80%, 85%, or 90% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) to any one of SEQ ID NOs: 37-69; and the immunoglobulin light chain variable region comprises an amino acid sequence with at least 80%, 85%, or 90% sequence identity (e.g., at least 91%, at least 92%, at least 93%, at least 94%, at least 95%, at least 96%, at least 97%, at least 98%, at least 99%, or 100% sequence identity) to any of SEQ ID NOs: 70-77. In some embodiments, the immunoglobulin heavy chain variable region comprises the CDRs of any of SEQ ID NOs: 37-69 and has an amino acid sequence with at least 80%, 85%, or 90% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) thereto; and the immunoglobulin light chain variable region comprises the CDRs of any of SEQ ID NOs: 70-77 and has an amino acid sequence with at least 80%, 85%, or 90% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) thereto; wherein the CDRs are as determined according to Kabat, Chothia, Martin (Enhanced Chothia), IGMT, or AHo.

Also provided herein is a CD122 binding agent comprising an immunoglobulin heavy chain and an immunoglobulin light chain, wherein, the immunoglobulin heavy chain variable region comprises SEQ ID NO: 69; an amino acid sequence with at least 80%, 85%, or 90% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) to SEQ ID NO; 69; and/or at least the CDRs of SEQ ID NO: 69, wherein the CDR regions are as determined in accordance with Kabat, Chothia, Martin (Enhanced Chothia), IGMT, or AHo; and the immunoglobulin light chain variable region comprises SEQ ID NO: 77; an amino acid sequence with at least 80%, 85%, or 90% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) to SEQ ID NO: 77; and/or at least the CDR regions of SEQ ID NO: 77, wherein the CDR regions are as determined by Kabat, Chothia, Martin (Enhanced Chothia), IGMT, or AHo.

In some embodiments, the CD122 binding agent comprises a heavy chain variable region of SEQ ID NO: 69 and light chain variable region of SEQ ID NO: 77, or at least the CDRs thereof as determined by Kabat. In some embodiments, the antibody comprises a heavy chain variable region of SEQ ID NO: 69 and light chain variable region of SEQ ID NO: 77, or at least the CDRs thereof as determined by Chothia. In some embodiments, the antibody comprises a heavy chain variable region of SEQ ID NO: 69 and light chain variable region of SEQ ID NO: 77, or at least the CDRs thereof as determined by Martin. In some embodiments, the antibody comprises a heavy chain variable region of SEQ ID NO: 69 and light chain variable region of SEQ ID NO: 77, or at least the CDRs thereof as determined by IGMT. In some embodiments, the antibody comprises a heavy chain variable region of SEQ ID NO: 69 and light chain variable region of SEQ ID NO: 77, or at least the CDRs thereof as determined by AHo. By way of further example, the CD122 binding agent can comprise an immunoglobulin heavy chain comprising SEQ ID NO: 115 and an immunoglobulin light chain comprising SEQ ID NO: 116, or an amino acid sequence with at least 80%, 85%, or 90% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) to SEQ ID NO: 115 and 116, respectively, optionally wherein the sequence retains the heavy chain and light chain CDRs of SEQ ID NO: 115 and 116, respectively, wherein the CDRs are as determined in accordance with any of the various known immunoglobulin numbering schemes (e.g., Kabat, Chothia, Martin (Enhanced Chothia), IGMT, or AHo).

Also provided herein is a CD122 binding agent comprising an immunoglobulin heavy chain and an immunoglobulin light chain, wherein, the immunoglobulin heavy chain variable region comprises SEQ ID NO: 68; an amino acid sequence with at least 80%, 85%, or 90% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) to SEQ ID NO: 68; and/or at least the CDRs of SEQ ID NO: 68, wherein the CDR regions are as determined in accordance with Kabat, Chothia, Martin (Enhanced Chothia), IGMT, or AHo; and the immunoglobulin light chain variable region comprises SEQ ID NO: 77; an amino acid sequence with at least 80%, 85%, or 90% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) to SEQ ID NO: 77; and/or at least the CDR regions of SEQ ID NO: 77, wherein the CDR regions are as determined by Kabat, Chothia, Martin (Enhanced Chothia), IGMT, or AHo.

In some embodiments, the CD122 binding agent comprises a heavy chain variable region of SEQ ID NO: 68 and light chain variable region of SEQ ID NO: 77, or at least the CDRs thereof as determined by Kabat. In some embodiments, the antibody comprises a heavy chain variable region of SEQ ID NO: 68 and light chain variable region of SEQ ID NO: 77, or at least the CDRs thereof as determined by Chothia. In some embodiments, the antibody comprises a heavy chain variable region of SEQ ID NO: 68 and light chain variable region of SEQ ID NO: 77, or at least the CDRs thereof as determined by Martin. In some embodiments, the antibody comprises a heavy chain variable region of SEQ ID NO: 68 and light chain variable region of SEQ ID NO: 77, or at least the CDRs thereof as determined by IGMT. In some embodiments, the antibody comprises a heavy chain variable region of SEQ ID NO: 68 and light chain variable region of SEQ ID NO: 77, or at least the CDRs thereof as determined by AHo. By way of further example, the CD122 binding agent can comprise an immunoglobulin heavy chain comprising SEQ ID NO: 165 and an immunoglobulin light chain comprising SEQ ID NO: 116, or an amino acid sequence with at least 80%, 85%, or 90% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) to SEQ ID NO: 165 and 116, respectively, optionally wherein the sequence retains the heavy chain and light chain CDRs of SEQ ID NO: 165 and 116, respectively, wherein the CDRs are as determined in accordance with any of the various known immunoglobulin numbering schemes (e.g., Kabat, Chothia, Martin (Enhanced Chothia), IGMT, or AHo).

In yet another aspect, the disclosure provides a CD122 binding agent comprising an immunoglobulin heavy chain variable region and immunoglobulin light chain variable region, wherein the immunoglobulin heavy chain and light chain variable regions of the CD122 binding agent comprises the following CDRs when determined in accordance with Kabat numbering: a CDRH1 comprising SEQ ID NO: 108; a CDRH2 comprising SEQ ID NO: 109; a CDRH3 comprising SEQ ID NO: 98; a CDRL1 comprising SEQ ID NO: 110; a CDRL2 comprising SEQ ID NO: 111; and a CDRL3 comprising SEQ ID NO: 112.

In some embodiments, the immunoglobulin heavy and light chain variable regions comprises the following CDRs when determined in accordance with Kabat numbering: CDRH1 comprises any one of SEQ ID NOs: 78-93; CDRH2 comprises any one of SEQ ID NOs: 94-97; CDRH3 comprises SEQ ID NO: 98; CDRL1 comprises any one of SEQ ID NOs: 99-102; CDRL2 comprises SEQ ID NO: 103 or 104; and CDRL3 comprises any one of SEQ ID NOs: 105-107.

In another aspect, the disclosure provides a CD122 binding agent comprising an immunoglobulin heavy chain and an immunoglobulin light chain, wherein, the immunoglobulin heavy chain variable region comprises SEQ ID NO: 150, or at least the CDR regions thereof, and the immunoglobulin light chain variable region comprises SEQ ID NO: 151, or at least the CDR regions thereof, wherein the CDR regions are as determined by Kabat, Chothia, Martin (Enhanced Chothia), IGMT, or AHo.

Also provided is a CD122 binding agent comprising an immunoglobulin heavy chain variable region and an immunoglobulin light chain variable region, wherein the immunoglobulin heavy chain variable region comprises any one of SEQ ID NOs: 1-24 or at least the CDRs thereof; and the immunoglobulin light chain variable region comprises any one of SEQ ID NOs: 25-36 or at least the CDRs thereof, wherein the CDR regions are as determined by Kabat, Chothia, Martin (Enhanced Chothia), IGMT, or AHo.

Also provided herein is a CD122 binding agent comprising an immunoglobulin heavy chain variable region and an immunoglobulin light chain variable region, wherein the immunoglobulin heavy chain variable region comprises an amino acid sequence with at least; 80%, 85%, or 90% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) to any one of SEQ ID NOs: 1-24; and the immunoglobulin light chain variable region comprises at least 80%, 85%, or 90% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) to any one of SEQ ID NOs: 25-36. In some embodiments, the immunoglobulin heavy chain variable region comprises the CDRs of any of SEQ ID NOs: 1-24 and has an amino acid sequence with at least 80%, 85%, or 90% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) thereto; and the immunoglobulin light chain variable region comprises the CDRs of any of SEQ ID NOs: 25-36, and has an amino acid sequence with at least 80%, 85%, or 90% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) thereto; wherein the CDRs are as determined according to Kabat, Chothia, Martin (Enhanced Chothia), IGMT, or AHo.

Also provided is a CD122 binding agent comprising an immunoglobulin heavy chain and an immunoglobulin light chain, wherein the immunoglobulin heavy chain variable region comprises SEQ ID NO: 24; an amino acid sequence with at least 80%, 85%, or 90% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) to SEQ ID NO: 24; and/or at least the CDR regions of SEQ ID NO: 24, wherein the CDR regions are as determined in accordance with Kabat, Chothia, Martin (Enhanced Chothia), IGMT, or AHo; and the immunoglobulin light chain variable region comprises SEQ ID NO: 35; an amino acid sequence with at least 80%, 85%, or 90% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) to SEQ ID NO: 35; and/or at least the CDR regions thereof, wherein the CDR regions are as determined by Kabat, Chothia, Martin (Enhanced Chothia), IGMT, or AHo.

In some embodiments, the CD122 binding agent comprises a heavy chain variable region of SEQ ID NO: 24 and light chain variable region of SEQ ID NO: 35, or at least the CDRs thereof as determined by Kabat. In some embodiments, the antibody comprises a heavy chain variable region of SEQ ID NO: 24 and light chain variable region of SEQ ID NO: 35, or at least the CDRs thereof as determined by Chothia. In some embodiments, the antibody comprises a heavy chain variable region of SEQ ID NO: 24 and light chain variable region of SEQ ID NO: 35, or at least the CDRs thereof as determined by Martin. In some embodiments, the antibody comprises a heavy chain variable region of SEQ ID NO: 24 and light chain variable region of SEQ ID NO: 35, or at least the CDRs thereof as determined by IGMT. In some embodiments, the antibody comprises a heavy chain variable region of SEQ ID NO: 24 and light chain variable region of SEQ ID NO: 35, or at least the CDRs thereof as determined by AHo. By way of further example, the CD122 binding agent can comprise an immunoglobulin heavy chain comprising SEQ ID NO: 152 and an immunoglobulin light chain comprising SEQ ID NO: 153, or an amino acid sequence with at least 80%, 85%, or 90% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) to SEQ ID NOs: 152 and 153, optionally wherein the sequence retains the heavy chain and light chain CDRs of SEQ ID NOs: 152 and 153 as determined in accordance with any of the various known immunoglobulin numbering schemes (e.g., Kabat, Chothia, Martin (Enhanced Chothia), IGMT, or AHo).

Also provided is a CD122 binding agent comprising an immunoglobulin heavy chain and an immunoglobulin light chain, wherein the immunoglobulin heavy chain variable region comprises SEQ ID NO: 23; an amino acid sequence with at least 80%, 85%, or 90% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) to SEQ ID NO: 23; and/or at least the CDR regions of SEQ ID NO: 23, wherein the CDR regions are as determined in accordance with Kabat, Chothia, Martin (Enhanced Chothia), IGMT, or AHo; and the immunoglobulin light chain variable region comprises SEQ ID NO: 35; an amino acid sequence with at least 80%, 85%, or 90% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) to SEQ ID NO: 35; and/or at least the CDR regions thereof, wherein the CDR regions are as determined by Kabat, Chothia, Martin (Enhanced Chothia), IGMT, or AHo.

In some embodiments, the CD122 binding agent comprises a heavy chain variable region of SEQ ID NO: 23 and light chain variable region of SEQ ID NO: 35, or at least the CDRs thereof as determined by Kabat. In some embodiments, the antibody comprises a heavy chain variable region of SEQ ID NO: 23 and light chain variable region of SEQ ID NO: 35, or at least the CDRs thereof as determined by Chothia. In some embodiments, the antibody comprises a heavy chain variable region of SEQ ID NO: 23 and light chain variable region of SEQ ID NO: 35, or at least the CDRs thereof as determined by Martin. In some embodiments, the antibody comprises a heavy chain variable region of SEQ ID NO: 23 and light chain variable region of SEQ ID NO: 35, or at least the CDRs thereof as determined by IGMT. In some embodiments, the antibody comprises a heavy chain variable region of SEQ ID NO: 23 and light chain variable region of SEQ ID NO: 35, or at least the CDRs thereof as determined by AHo. By way of further example, the CD122 binding agent can comprise an immunoglobulin heavy chain comprising SEQ ID NO: 157 and an immunoglobulin light chain comprising SEQ ID NO: 153, or an amino acid sequence with at least 80%, 85%, or 90% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) to SEQ ID NOs: 157 and 153, optionally wherein the sequence retains the heavy chain and light chain CDRs of SEQ ID NOs: 157 and 153 as determined in accordance with any of the various known immunoglobulin numbering schemes (e.g., Kabat, Chothia, Martin (Enhanced Chothia), IGMT, or AHo).

Also provided is a CD122 binding agent comprising an immunoglobulin heavy chain and an immunoglobulin light chain, wherein the immunoglobulin heavy chain variable region comprises SEQ ID NO: 16; an amino acid sequence with at least 80%, 85%, or 90% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) to SEQ ID NO: 16; and/or at least the CDR regions of SEQ ID NO: 16, wherein the CDR regions are as determined in accordance with Kabat, Chothia, Martin (Enhanced Chothia), IGMT, or AHo; and the immunoglobulin light chain variable region comprises SEQ ID NO: 28; an amino acid sequence with at least 80%, 85%, or 90% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) to SEQ ID NO: 28; and/or at least the CDR regions thereof, wherein the CDR regions are as determined by Kabat, Chothia, Martin (Enhanced Chothia), IGMT, or AHo.

In some embodiments, the CD122 binding agent comprises a heavy chain variable region of SEQ ID NO: 16 and light chain variable region of SEQ ID NO: 28, or at least the CDRs thereof as determined by Kabat. In some embodiments, the antibody comprises a heavy chain variable region of SEQ ID NO: 16 and light chain variable region of SEQ ID NO: 28, or at least the CDRs thereof as determined by Chothia. In some embodiments, the antibody comprises a heavy chain variable region of SEQ ID NO: 16 and light chain variable region of SEQ ID NO: 28, or at least the CDRs thereof as determined by Martin. In some embodiments, the antibody comprises a heavy chain variable region of SEQ ID NO: 16 and light chain variable region of SEQ ID NO: 28, or at least the CDRs thereof as determined by IGMT. In some embodiments, the antibody comprises a heavy chain variable region of SEQ ID NO: 16 and light chain variable region of SEQ ID NO: 28, or at least the CDRs thereof as determined by AHo. By way of further example, the CD122 binding agent can comprise an immunoglobulin heavy chain comprising SEQ ID NO: 159 and an immunoglobulin light chain comprising SEQ ID NO: 161, or an amino acid sequence with at least 80%, 85%, or 90% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) to SEQ ID NOs: 159 and 161, optionally wherein the sequence retains the heavy chain and light chain CDRs of SEQ ID NOs: 159 and 161 as determined in accordance with any of the various known immunoglobulin numbering schemes (e.g., Kabat, Chothia, Martin (Enhanced Chothia), IGMT, or AHo).

According to another aspect, the disclosure provides a CD122 binding agent comprises heavy and light chain immunoglobulin variable regions with the following CDRs when determined by Kabat numbering: a CDRH1 comprising SEQ ID NO: 146; a CDRH2 comprising SEQ ID NO: 147; and a CDRH3 comprising SEQ ID NO: 148; a CDRL1 comprising SEQ ID NO: 138; a CDRL2 comprising SEQ ID NO: 139; and a CDRL3 comprising SEQ ID NO: 149. In some embodiments, the CDRH1 comprises any of SEQ ID NOs: 117-126; CDRH2 comprises any one of SEQ ID NOs: 127-135; CDRH3 comprises SEQ ID NO: 136 or 137; CDRL1 comprises SEQ ID NO: 138, CDRL2 comprises SEQ ID NO: 139; and CDRL3 comprises any of SEQ ID NOs: 140-145.

Further provided is a CD122 binding agent comprising the heavy and light chain variable regions set forth in Table 1A or 1B or at least the CDRs thereof as determined Kabat, Chothia, Martin (Enhanced Chothia), IGMT, or AHo. Also provided is a CD122 binding agent comprising the CDR regions set forth in Table 1C or 1D when determined according to Kabat numbering.

Sequence “identity,” as described herein, can be determined by comparing a nucleic acid or amino acid sequence of interest to a reference nucleic acid or amino acid sequence. The percent identity is the number of nucleotides or amino acid residues that are the same (i.e., that are identical) as between the sequence of interest and the reference sequence divided by the length of the longest sequence (i.e., the length of either the sequence of interest or the reference sequence, whichever is longer). A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3×, FAS™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Nall. Acad. Sci. USA, 106(10): 3770-3775 (2009), Durbin et al., eds., Biological Sequence Analysis: Probalistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

Variation in sequence identity can be accomplished through addition, substitution, or deletion of one or more amino acid residues. An amino acid “replacement” or “substitution” refers to the replacement of one amino acid at a given position or residue by another amino acid at the same position or residue within a polypeptide sequence. The amino acid replacement or substitution can be conservative, semi-conservative, or non-conservative depending upon whether the substitution is by an amino acid residue that has similar properties to the residue being replaced. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra).

Amino acids can be broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or Ile), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gln), lysine (K or Lys), and arginine (R or Arg).

Aliphatic amino acids may be sub-divided into four sub-groups. The “large aliphatic non-polar sub-group” consists of valine, leucine, and isoleucine. The “aliphatic slightly-polar sub-group” consists of methionine, serine, threonine, and cysteine. The “aliphatic polar/charged sub-group” consists of glutamic acid, aspartic acid, asparagine, glutamine, lysine, and arginine. The “small-residue sub-group” consists of glycine and alanine. The group of charged/polar amino acids may be sub-divided into three sub-groups: the “positively-charged sub-group” consisting of lysine and arginine, the “negatively-charged sub-group” consisting of glutamic acid and aspartic acid, and the “polar sub-group” consisting of asparagine and glutamine.

Aromatic amino acids may be sub-divided into two sub-groups: the “nitrogen ring sub-group” consisting of histidine and tryptophan and the “phenyl sub-group” consisting of phenylalanine and tyrosine.

Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free —OH can be maintained, and glutamine for asparagine such that a free —NH2 can be maintained. “Semi-conservative mutations” include amino acid substitutions of amino acids within the same groups listed herein, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

In some embodiments, the CD122 binding agent can comprise, consist essentially of, or consist of the immunoglobulin heavy and light chain variable region or full heavy and light chain polypeptides provided herein. The CD122 binding agent can be any type of molecule or construct comprising at least the specified immunoglobulin heavy and light chain variable regions. Thus, the CD122 binding agent can be, for instance, a whole immunoglobulin or antibody, as described herein, or an antigen-binding (CD122 binding) immunoglobulin or antibody “fragment.” The term “fragment” used with respect to an antibody or immunoglobulin means any molecule or construct that comprises some part of an immunoglobulin or antibody and binds the target antigen. Such a fragment will generally comprise at least the parts of the heavy and light chain variable regions including the CDRs, and may also include parts of the constant regions, optionally along with other elements that are not normally part of an immunoglobulin or antibody (e.g., linkers, etc.). Examples of such “fragments” include, but are not limited to, (i) a Fab fragment, which is a monovalent fragment consisting of the V_{L}, V_{H}, C_{L}, and CH₁ domains, (ii) a F(ab′)₂ fragment, which is a bivalent fragment comprising two Fab fragments linked by a disulfide bridge at the hinge region, (iii) a Fv fragment consisting of the V_{L} and V_{H} domains of a single arm of an antibody, (iv) a Fab′ fragment, which results from breaking the disulfide bridge of an F(ab′)₂ fragment using mild reducing conditions; (v) a diabody; (vi) a single-chain variable region (scFv), and (vii) a disulfide-stabilized Fv fragment (dsFv).

In some embodiments, the CD122 binding agent comprises an immunoglobulin heavy chain constant region, such as a fragment crystallizable (Fe) region or portion thereof. The Fc region can be of any Ig class/subclass (IgA (IgA1, IgA2), IgD, IgE, IgG (IgG1, IgG2, IgG3 and IgG4), IgM, including variants thereof. In a particular embodiment, the CD122 binding agent comprises an Fc region that binds an Fc receptor of an antigen-presenting cell (e.g., dendritic cell, macrophage, Langerhans cell, or B cell). The Fc receptor can be an Fcγ receptor (FcγR), such as FcγRI (CD64), FcγRIIA (CD32), FcγRIIB (CD32), FcγRIIIA (CD16a), FcγRIIIB (CD16b). In one embodiment, the CD122 binding agent comprises an Fc region that binds FcγR, such as IgG1. Thus, in some embodiments, the CD122 binding agent is a “whole” or “complete” Ig (i.e., an antibody). In additional embodiments, the CD122 binding agent is an IgG antibody, particularly an IgG1 antibody. In additional embodiments, the CD122 agent is an IgG antibody, particularly an IgG4 antibody.

In some embodiments, the CD122 binding agent comprises an Fc region that has reduced (including substantially or completely abolished) binding to one or more (or all) Fcγ receptors and, thus, reduced immune effector function. The CD122 binding agent can comprise, for example, an IgG Fc region (e.g., an IgG1 or IgG4) with mutations that reduce effector functions, for instance removal of the Fc N-linked glycosylation site in human IgG1, leucine to glutamic acid substitution at position 235 of IgG1 Fc, or other modifications of the hinge region positions 234-237; double mutation Leu234Ala and Leu235Ala (“LALA” mutation); substitution P329G in IgG1; or both P329G and LALA mutations. In some embodiments, the CD122 binding agent comprises an IgG1 Fc with the LALA mutation, alone or together with P329G.

The isolated CD122-binding agent also can be an antibody conjugate. In this respect, the isolated CD122-binding agent can be a conjugate comprising the CD122-binding agent (e.g., anti-CD122 antibody or antibody fragment) and another biologically active moiety. For example, the CD122 binding agent can be conjugated to a peptide, a fluorescent molecule, or a chemotherapeutic agent, particularly an agent useful in suppressing an immune response.

The CD122 binding agent can be, or can be obtained from, a human antibody, a non-human antibody, or a chimeric antibody. By “chimeric” is meant an antibody or fragment thereof comprising both human and non-human regions. Preferably, the isolated CD122 binding agent is a humanized antibody. A “humanized” antibody is a monoclonal antibody comprising a human antibody scaffold and at least one CDR obtained or derived from a non-human antibody. Non-human antibodies include antibodies isolated from any non-human animal, such as, for example, a rodent (e.g., a mouse or rat). A humanized antibody can comprise, one, two, or three CDRs obtained or derived from a non-human antibody. In a preferred embodiment of the invention, CDRH3 of the CD122 binding agent is obtained or derived from a mouse monoclonal antibody, while the remaining variable regions and constant region of the inventive CD122 binding agent are obtained or derived from a human monoclonal antibody.

A human antibody, a non-human antibody, a chimeric antibody, or a humanized antibody can be obtained by any means, including via in vitro sources (e.g., a hybridoma or a cell line producing an antibody recombinantly) and in vivo sources (e.g., rodents). Methods for generating antibodies are known in the art and are described in, for example, Köhler and Milstein, Eur. J. Immunol., 5: 511-519 (1976); Harlow and Lane (eds.), Antibodies: A Laboratory Manual, CSH Press (1988); and Janeway et al. (eds.), Immunobiology, 5th Ed., Garland Publishing, New York, NY (2001); Starkie et al., PLoS One, 11(3): e0152282 (2016)). In certain embodiments, a human antibody or a chimeric antibody can be generated using a transgenic animal (e.g., a mouse) wherein one or more endogenous immunoglobulin genes are replaced with one or more human immunoglobulin genes. Examples of transgenic mice wherein endogenous antibody genes are effectively replaced with human antibody genes include, but are not limited to, the Medarex HUMAB-MOUSE™, the Kirin TC MOUSE™, and the Kyowa Kirin KM-MOUSE™ (see, e.g., Lonberg, Nat. Biotechnol., 23(9): 1117-25 (2005), and Lonberg, Handb. Exp. Pharmacol., 181: 69-97 (2008)). A humanized antibody can be generated using any suitable method known in the art (see, e.g., An, Z. (ed.), Therapeutic Monoclonal Antibodies: From Bench to Clinic, John Wiley & Sons, Inc., Hoboken, New Jersey (2009)), including, e.g., grafting of non-human CDRs onto a human antibody scaffold (see, e.g., Kashmiri et al., Methods, 36(1): 25-34 (2005); and Hou et al., J. Biochem., 144(1): 115-120 (2008)). In one embodiment, a humanized antibody can be produced using the methods described in, e.g., U.S. Patent Application Publication 2011/0287485 A1.

The CD122 binding agent can have any suitable affinity for human CD122. The term “affinity” refers to the equilibrium constant for the reversible binding of two agents and is expressed as the dissociation constant (K_{D}). Affinity of a binding agent to a ligand, such as affinity of an antibody for an epitope, can be, for example, from about 1 picomolar (pM) to about 100 micromolar (μM) (e.g., from about 1 picomolar (pM) to about 1 nanomolar (nM), from about 1 nM to about 1 micromolar (μM), or from about 1 μM to about 100 μM). In one embodiment, the CD122 binding agent can bind to a CD122 protein with a K_{D} less than or equal to 1.5 nM (e.g., 1.4 nM, 1.3 nM, 1.2 nM, 1.0 nM, 0.9 nM, 0.8 nM, 0.7 nM, 0.6 nM, 0.5 nM, 0.4 nM, 0.3 nM, 0.2 nM, 0.1 nM, 0.05 nM, 0.025 nM, 0.01 nM, 0.001 nM, or a range defined by any two of the foregoing values). In another embodiment, the CD122 binding agent can bind to CD122 with a K_{D} less than or equal to 200 pM (e.g., 190 pM, 175 pM, 150 pM, 125 pM, 110 pM, 100 pM, 90 pM, 80 pM, 75 pM, 60 pM, 50 pM, 40 pM, 30 pM, 25 pM, 20 pM, 15 pM, 10 pM, 5 pM, 1 pM, or a range defined by any two of the foregoing values). In some embodiments, the CD122 binding agent is cross-reactive with cynomolgus CD122 with an affinity in any of the foregoing ranges discussed with respect to human CD122. Immunoglobulin affinity for an antigen or epitope of interest can be measured using any art-recognized assay. Such methods include, for example, fluorescence activated cell sorting (FACS), separable beads (e.g., magnetic beads), surface plasmon resonance (SPR), solution phase competition (KinExA®), antigen panning, and/or ELISA (see, e.g., Janeway et al. (eds.), Immunobiology, 5th ed., Garland Publishing, New York, NY, 2001).

IL-2Rβ (CD122) is the common beta receptor subunit shared for IL-15 and IL-2. IL-15 and IL-2 signaling mediate the survival and maintenance of tissue resident memory T cells (TRM) and NK cell subsets. The presence of long-lived and persistent TRM have been shown to be pathogenic drivers of tissue-specific immune-mediated inflammation, present in the skin of dermatologic diseases, where defined borders of inflammation often recur. TRM are also observed in other tissue-specific inflammatory disorders including gastroenterology and rheumatology.

In some embodiments, the CD122 binding agent binds CD122 and at least partially (or completely) inhibits IL-15 signaling. Alternatively or in addition, the CD122 binding agent binds CD122 and at least partially (or completely) inhibits IL-2 signaling. For instance, in some embodiments, the CD122 binding agent inhibits IL-15 signaling at least partially (or completely) and inhibits IL-2 signaling through the low affinity IL-2 receptor (comprised of CD122 and the common gamma subunit, CD132), without eliminating (or without inhibiting) IL-2 signaling through the high affinity IL-2 receptor (comprised of CD122, CD132 and the alpha receptor subunit for IL-2, CD25). The CD122 binding agent can bind CD122 on any CD122-expressing cell type, such as regulatory T-cells (Tregs), memory T-cells, CD56+ T cells (NKT cells), innate lymphocytes (ILCs), and gamma delta T cells. Without wishing to be bound by any particular theory or mechanism of action, it is believed that the inhibition of IL-15 signaling (in whole or in part) and inhibition of IL-2 signaling (in whole or in part) through the low affinity IL-2 receptor expressed on NK cells and T cells, without eliminating IL-2 signaling through the high affinity IL-2 receptor expressed on regulatory T cells, is believed to provide additional anti-inflammatory benefit by reducing NK cell and pathogenic T cell numbers while sparing or potentially enhancing regulatory T cell numbers.

### Methods of Use/Treatment

The CD122 binding agents are believed to be particularly useful for inhibiting IL-15 signaling and inhibiting IL-2 signaling, particularly though the low affinity IL-2 receptor (comprised of CD122 and the common gamma subunit, CD132). Thus, the binding agents can be used in method of treating a disease or disorder, or symptom thereof, caused by or facilitated through IL-15 signaling or IL-2 signaling, especially though the low affinity IL-2 receptor. For instance, the CD122 binding agent can be used to inhibit an inflammatory or immune response or treat a disease or disorder associated therewith, particularly one characterized by pathogenic memory T-cells (including tissue-resident memory T-cells (TRM cells)) and NK cells. In some embodiments, signaling to regulatory T cells (Tregs) is preserved in whole or in-part (i.e., Treg signaling is not entirely prevented, or is not significantly inhibited, or not inhibited at all in the presence of the CD122 binding agent as compared to such signaling in the absence of the CD122 binding agent). Negative regulation of the immune system as used herein in synonymous with immunosuppression. It will be appreciated that the CD122 binding agent can be administered prior to the onset of symptoms in some instances (e.g., prior to exposure to an antigen that triggers an immune response) so as to prevent, suppress, or reduce the severity of an immune response upon introduction of the antigen.

In some aspects, the CD122 binding agent can be used to treat an inflammatory or autoimmune disorder or otherwise inhibit or suppress an immune or inflammatory response. Examples of inflammatory or autoimmune disorders include, for example, infections (viral, bacterial, fungal and parasitic), endotoxic shock associated with infection, arthritis, rheumatoid arthritis, including TNF-refractory rheumatoid arthritis, Sjogren's Syndrome, asthma, Chronic obstructive pulmonary disease (COPD), pelvic inflammatory disease, Behcet disease, Alzheimer's Disease, inflammatory bowel disease including Crohn's disease and ulcerative colitis, Peyronie's Disease, celiac disease, gallbladder disease, Pilonidal disease, peritonitis, psoriasis, psoriatic arthritis, contact hypersensitivity, allergic disease, eosinophilia, eosinophilic esophagitis, vasculitis, antineutrophil cytoplasmic antibody-associated (ANCA) vasculitis, surgical adhesions, stroke, Type I Diabetes, lyme disease, arthritis, meningoencephalitis, autoimmune or noninfectious uveitis, immune mediated inflammatory disorders of the central and peripheral nervous system such as multiple sclerosis, lupus (such as systemic lupus erythematosus and chronic discoid lupus erythematosus) and Guillain-Barr syndrome, Atopic dermatitis, polymyositis, dermatomyositis, autoimmune hepatitis, fibrosing alveolitis, Grave's disease, IgA nephropathy, idiopathic thrombocytopenic purpura, Meniere's disease, pemphigus, pemphigoid, primary biliary cholangitis, hepatitis, sarcoidosis, scleroderma (localized scleroderma, systemic scleroderma, and progressive systemic scleroderma), Ganulomatosis with polyangiitis, other autoimmune disorders, cholangitis, pancreatitis, trauma (surgery), graft-versus-host disease, transplant rejection, heart disease including ischaemic diseases such as myocardial infarction as well as atherosclerosis, periarteritis nodosa (polyarteritis nodosa and microscopic polyangiitis), allergic granulomatous angiitis, hypersensitivity angiitis, aortitis syndrome (Takayasu arteritis), temporal arteritis, intravascular coagulation, bone resorption, osteoporosis, osteoarthritis, periodontitis, including chronic periodontitis, and hypochlorhydia, Still's disease, Cogan's syndrome, RS3PE, polymyalgia rheumatica, fibromyalgia syndrome, antiphospholipid antibody syndrome, eosinophilic fasciitis, Guillain-Barre syndrome, myasthenia gravis, chronic atrophic gastritis, Goodpasture's syndrome, rapidly progressive glomerulonephritis, megaloblastic anemia, hemolytic anemia, autoimmune neutropenia, Hashimoto's thyroiditis, autoimmune adrenal insufficiency, primary hypothyroidism, idiopathic Addison's disease (chronic adrenal insufficiency), herpes gestationis, linear IgA bullous skin disease, epidermolysis bullosa acquisita, alopecia areata, vitiligo, Harada disease, autoimmune optic neuropathy, idiopathic azoospermia, recurrent fetal loss (e.g., recurrent miscarriage or abortion), infertility related to lack of fetal-maternal tolerance, or respiratory diseases, such as asthma and ARDS (acute respiratory distress syndrome).

In some embodiments, the disease or disorder is psoriasis, psoriatic arthritis, rheumatoid arthritis, contact hypersensitivity, allergic disease, eosinophilia, eosinophilic esophagitis, respiratory diseases such as asthma and ARDS (acute respiratory distress syndrome), Giant Cell Arteritis, Polymyalgia Rheumatica, Primary Sjögren's Syndrome, TNF-refractory Rheumatoid Arthritis, Alopecia Areata, Primary Biliary Cholangitis (PBC), Graft vs Host Disease (GvHD), Vitiligo, ANCA Vasculitis, Type 1 Diabetes, inflammatory bowel disease including Crohn's disease and ulcerative colitis, asthma, chronic periodontitis, recurrent fetal loss, celiac disease, transplant rejection or Noninfectious Uveitis.

An “immune response” can entail, for example, antibody production and/or the activation of immune effector cells (e.g., T-cells), production of inflammatory cytokines, or any of the indications or disorders described herein or otherwise known in the art. As used herein, the terms “treatment,” “treating,” and the like refer to obtaining a desired pharmacologic and/or physiologic effect. Preferably, the effect is therapeutic, i.e., the effect partially or completely reduces an adverse symptom associated with IL-2 signaling or a disease associated therewith. To this end, the inventive method comprises administering a “therapeutically effective amount” of the CD122-binding agent. A “therapeutically effective amount” refers to an amount effective, at dosages and for periods of time necessary, to achieve a desired therapeutic result. The therapeutically effective amount may vary according to factors such as the disease state, age, sex, and weight of the individual, and the ability of the CD122-binding agent to elicit a desired response in the individual. The pharmacologic and/or physiologic effect may be prophylactic, i.e., the effect completely or partially prevents an adverse symptom or disease associated with IL-2 signaling.

The CD122 binding agent can be part of a composition suitable for administration to a mammal. Preferably, the composition is a pharmaceutically acceptable (e.g., physiologically acceptable) composition, which comprises a carrier, preferably a pharmaceutically acceptable (e.g., physiologically acceptable) carrier, and the inventive amino acid sequences, antigen-binding agent, or vector. Any suitable carrier can be used within the context of the invention, and such carriers are well known in the art. The choice of carrier will be determined, in part, by the particular site to which the composition may be administered and the particular method used to administer the composition. The composition also can comprise any other excipient used in the formulation of therapeutic molecules (e.g., proteins or antibodies), particularly parenteral formulations, including, for instance, buffers, tonicity modifiers, stabilizers, surfactants and the like. The composition optionally can be sterile. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, PA (2001).

The dosage used will depend on the particular circumstances in which the binding agent is employed. A typical dose of the CD122 binding agent can be, for example, in the range of 1 μg/kg to 100 mg/kg of animal or human body weight; however, doses below or above this exemplary range are within the scope of the invention. Therapeutic or prophylactic efficacy can be monitored by periodic assessment of treated patients. For repeated administrations over several days or longer, depending on the condition, the treatment can be repeated until a desired suppression of disease symptoms occurs. However, other dosage regimens may be useful and are within the scope of the invention. The desired dosage can be delivered by a single bolus administration of the composition, by multiple bolus administrations of the composition, or by continuous infusion administration of the composition.

The CD122 binding agent can be administered to a mammal using standard administration techniques, including oral, intravenous, intraperitoneal, subcutaneous, pulmonary, transdermal, intramuscular, intranasal, buccal, sublingual, or suppository administration. The composition preferably is suitable for parenteral administration. The term “parenteral,” as used herein, includes intravenous, intramuscular, subcutaneous, rectal, vaginal, and intraperitoneal administration. More preferably, the composition is administered to a mammal using peripheral systemic delivery by intravenous, intraperitoneal, or subcutaneous injection.

Once administered to a mammal (e.g., a human), the biological activity of the inventive CD122 binding agent can be measured by any suitable method known in the art. For example, the biological activity can be assessed by determining the stability of a particular CD122 binding agent. In one embodiment of the invention, the CD122 binding agent (e.g., an antibody) has an in vivo half-life between about 30 minutes and 45 days (e.g., about 30 minutes, about 45 minutes, about 1 hour, about 2 hours, about 4 hours, about 6 hours, about 10 hours, about 12 hours, about 1 day, about 5 days, about 10 days, about 15 days, about 25 days, about 35 days, about 40 days, about 45 days, or a range defined by any two of the foregoing values). In another embodiment, the CD122 binding agent has an in vivo half-life between about 2 hours and 20 days (e.g., about 5 hours, about 10 hours, about 15 hours, about 20 hours, about 2 days, about 3 days, about 7 days, about 12 days, about 14 days, about 17 days, about 19 days, or a range defined by any two of the foregoing values). In another embodiment, the CD122 binding agent has an in vivo half-life between about 10 days and about 40 days (e.g., about 10 days, about 13 days, about 16 days, about 18 days, about 20 days, about 23 days, about 26 days, about 29 days, about 30 days, about 33 days, about 37 days, about 38 days, about 39 days, about 40 days, or a range defined by any two of the foregoing values). In other embodiments, the biological activity of the inventive CD122 binding agent can be assessed by monitoring receptor occupancy on CD122-expressing cells in peripheral blood and tissues, such as skin tissues. In other embodiments, the biological activity of the inventive CD122-binding agent can be assessed by monitoring reduction of immune cells that express CD122, such as NK cells.

The CD122-binding agent of the invention may be administered alone or in combination with other active agents or drugs. For example, the CD122-binding agent can be administered in combination with other agents for the treatment or prevention of the diseases disclosed herein. In this respect, the CD122-binding agent can be used in combination with at least one other inflammatory or autoimmune disorder-inhibiting agent including, for example, other monoclonal antibodies, disease-killing viruses, gene therapy, cytokine therapy and adoptive T-cell transfer, and/or surgery. In some embodiments, the CD122-binding agent is used in combination with (e.g., co-therapy; administered simultaneously or sequentially in any order, or in any dosing regimen) with a CD28 antagonist, such as a cytotoxic T-lymphocyte-associated protein 4 (CTLA4) (e.g., CTLA4-Ig, belatacept, or abatacept). The inventive CD122-binding agent described herein can also be used in combination with at least one other immunosuppressive agent, including, for example, methotrexate, corticosteroids, and other small molecule agents used to treat autoimmune and inflammatory disease. When the inventive method treats an infectious disease, the CD122-binding agent can be administered in combination with at least one anti-bacterial agent or at least one anti-viral agent. In this respect, the anti-bacterial agent can be any suitable antibiotic known in the art. The anti-viral agent can be any vaccine of any suitable type that specifically targets a particular virus (e.g., live-attenuated vaccines, subunit vaccines, recombinant vector vaccines, and small molecule anti-viral therapies (e.g., viral replication inhibitors and nucleoside analogs). In some embodiments, the CD122-binding agent is used in combination with (e.g., co-therapy; administered simultaneously or sequentially in any order, or in any dosing regimen) with a Janus kinase (JAK) inhibitor, for instance, abrocitinib, baricitinib, delgocitinib, fedratinib, filgotinib, oclacitinib, peficitinib, pacritinib, ruxolitinib, tofacitinib, or upadacitinib.

In addition to therapeutic uses, the CD122-binding agent described herein can be used in diagnostic or research applications. In this respect, the CD122-binding agent can be used in a method to diagnose a cancer or infectious disease. In a similar manner, the CD122-binding agent can be used in an assay to monitor CD122 protein levels in a subject being tested for a disease or disorder that is associated with abnormal CD122 expression. Research applications include, for example, methods that utilize the CD122-binding agent and a label to detect a CD122 protein in a sample, e.g., in a human body fluid or in a cell or tissue extract. The CD122-binding agent can be used with or without modification, such as covalent or non-covalent labeling with a detectable moiety. For example, the detectable moiety can be a radioisotope (e.g., 3H, ¹⁴C, ³P, ³¹S, or I), a fluorescent or chemiluminescent compound (e.g., fluorescein isothiocyanate, rhodamine, or luciferin), an enzyme (e.g., alkaline phosphatase, beta-galactosidase, or horseradish peroxidase), or prosthetic groups. Any method known in the art for separately conjugating an antigen-binding agent (e.g., an antibody) to a detectable moiety may be employed in the context of the invention (see, e.g., Hunter et al., Nature, 194: 495-496 (1962); David et al., Biochemistry, 13: 1014-1021 (1974); Pain et al., J. Immunol. Meth., 40: 219-230 (1981); and Nygren, J. Histochem. Cytochem., 30: 407-412 (1982)).

CD122 protein levels can be measured using the inventive CD122-binding agent by any suitable method known in the art. Such methods include, for example, radioimmunoassay (RIA), and FACS. Normal or standard expression values of CD122 protein can be established using any suitable technique, e.g., by combining a sample comprising, or suspected of comprising, a CD122 polypeptide with a CD122-specific antibody under conditions suitable to form an antigen-antibody complex. The antibody is directly or indirectly labeled with a detectable substance to facilitate detection of the bound or unbound antibody. Suitable detectable substances include various enzymes, prosthetic groups, fluorescent materials, luminescent materials, and radioactive materials (see, e.g., Zola, Monoclonal Antibodies: A Manual of Techniques, CRC Press, Inc. (1987)). The amount of PD-1 polypeptide expressed in a sample is then compared with a standard value.

The CD122-binding agent can be provided in a kit, i.e., a packaged combination of reagents in predetermined amounts with instructions for performing a diagnostic assay. If the CD122-binding agent is labeled with an enzyme, the kit desirably includes substrates and cofactors required by the enzyme (e.g., a substrate precursor which provides a detectable chromophore or fluorophore). In addition, other additives may be included in the kit, such as stabilizers, buffers (e.g., a blocking buffer or lysis buffer), and the like. The relative amounts of the various reagents can be varied to provide for concentrations in solution of the reagents which substantially optimize the sensitivity of the assay. The reagents may be provided as dry powders (typically lyophilized), including excipients which on dissolution will provide a reagent solution having the appropriate concentration.

### Nucleic Acids, Cells, Methods of Production

The invention also provides one or more isolated or purified nucleic acid sequences that encode the CD122 binding agent or individual heavy or light chain immunoglobulin polypeptides thereof. Thus, in one embodiment, the nucleic acid encodes an immunoglobulin light chain variable region or full immunoglobulin light chain as provided herein. In another embodiment, the nucleic acid encodes an immunoglobulin heavy chain variable region or full immunoglobulin heavy chain as provided herein. In yet another embodiment, the nucleic acid encodes both an immunoglobulin light chain variable region or full immunoglobulin light chain, and an immunoglobulin heavy chain variable region or full immunoglobulin heavy chain, as provided herein.

The terms “nucleic acid” and “nucleic acid sequence” are intended to encompass a polymer of DNA or RNA, i.e., a polynucleotide, which can be single-stranded or double-stranded and which can contain non-natural or altered nucleotides. The terms “nucleic acid” and “polynucleotide” as used herein refer to a polymeric form of nucleotides of any length, either ribonucleotides (RNA) or deoxyribonucleotides (DNA). These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, though not limited to, methylated and/or capped polynucleotides. Nucleic acids are typically linked via phosphate bonds to form nucleic acid sequences or polynucleotides, though many other linkages are known in the art (e.g., phosphorothioates, boranophosphates, and the like).

The nucleic acid can be part of a vector. The vector can be, for example, a plasmid, episome, cosmid, viral vector (e.g., retroviral or adenoviral), or phage. Suitable vectors and methods of vector preparation are well known in the art (see, e.g., Sambrook et al., Molecular Cloning, a Laboratory Manual, 3rd edition, Cold Spring Harbor Press, Cold Spring Harbor, N.Y. (2001), and Ausubel et al., Current Protocols in Molecular Biology, Greene Publishing Associates and John Wiley & Sons, New York, N.Y. (1994)).

In addition to the nucleic acid sequence encoding the immunoglobulin heavy and/or light chains, the vector can comprise expression control sequences, such as promoters, enhancers, polyadenylation signals, transcription terminators, internal ribosome entry sites (IRES), and the like, that provide for the expression of the coding sequence in a host cell. Exemplary expression control sequences are known in the art and described in, for example, Goeddel, Gene Expression Technology: Methods in Enzymology, Vol. 185, Academic Press, San Diego, Calif. (1990).

A large number of promoters, including constitutive, inducible, and repressible promoters, from a variety of different sources are well known in the art. Representative sources of promoters include for example, virus, mammal, insect, plant, yeast, and bacteria, and suitable promoters from these sources are readily available, or can be made synthetically, based on sequences publicly available, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, the RSV promoter. Inducible promoters include, for example, the Tet system (U.S. Pat. Nos. 5,464,758 and 5,814,618), the Ecdysone inducible system (No et al., Proc. Natl. Acad. Sci., 93: 3346-3351 (1996)), the T-REX™ system (Invitrogen, Carlsbad, CA), LACSWITCH™ system (Stratagene, San Diego, CA), and the Cre-ERT tamoxifen inducible recombinase system (Indra et al., Nuc. Acid. Res., 27: 4324-4327 (1999); Nuc. Acid. Res., 28: e99 (2000); U.S. Pat. No. 7,112,715; and Kramer & Fussenegger, Methods Mol. Biol., 308: 123-144 (2005)).

The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used CMV promoter) also comprise enhancer sequences. Enhancers can be located upstream, within, or downstream of coding sequences.

The vector also can comprise a selectable marker gene. The term “selectable marker gene,” as used herein, refers to a nucleic acid sequence that allow cells expressing the nucleic acid sequence to be specifically selected for or against, in the presence of a corresponding selective agent. Suitable selectable marker genes are known in the art and described in, e.g., International Patent Application Publications WO 1992/008796 and WO 1994/028143; Wigler et al., Proc. Natl. Acad. Sci. USA, 77: 3567-3570 (1980); O'Hare et al., Proc. Natl. Acad. Sci. USA, 78: 1527-1531 (1981); Mulligan & Berg, Proc. Natl. Acad. Sci. USA, 78: 2072-2076 (1981); Colberre-Garapin et al., J. Mol. Biol., 150: 1-14 (1981); Santerre et al., Gene, 30: 147-156 (1984); Kent et al., Science, 237: 901-903 (1987); Wigler et al., Cell, 11: 223-232 (1977); Szybalska & Szybalski, Proc. Natl. Acad. Sci. USA, 48: 2026-2034 (1962); Lowy et al., Cell, 22: 817-823 (1980); and U.S. Pat. Nos. 5,122,464 and 5,770,359.

In some embodiments, the vector is an “episomal expression vector” or “episome,” which is able to replicate in a host cell, and persists as an extrachromosomal segment of DNA within the host cell in the presence of appropriate selective pressure (see, e.g., Conese et al., Gene Therapy, 11: 1735-1742 (2004)). Representative commercially available episomal expression vectors include, but are not limited to, episomal plasmids that utilize Epstein Barr Nuclear Antigen 1 (EBNA1) and the Epstein Barr Virus (EBV) origin of replication (oriP). The vectors pREP4, pCEP4, pREP7, and pcDNA3.1 from Invitrogen (Carlsbad, CA) and pBK-CMV from Stratagene (La Jolla, CA) represent non-limiting examples of an episomal vector that uses T-antigen and the SV40 origin of replication in lieu of EBNA1 and oriP.

Other suitable vectors include integrating expression vectors, which may randomly integrate into the host cell's DNA, or may include a recombination site to enable the specific recombination between the expression vector and the host cell's chromosome. Such integrating expression vectors may utilize the endogenous expression control sequences of the host cell's chromosomes to effect expression of the desired protein. Examples of vectors that integrate in a site specific manner include, for example, components of the flp-in system from Invitrogen (Carlsbad, CA) (e.g., pcDNA™5/FRT), or the cre-lox system, such as can be found in the pExchange-6 Core Vectors from Stratagene (La Jolla, CA). Examples of vectors that randomly integrate into host cell chromosomes include, for example, pcDNA3.3 (when introduced in the absence of T-antigen) from ThermoFisher (Carlsbad, CA), UCOE from Millipore (Billerica, MA), and pCI or pFN10A (ACT) FLEXI™ from Promega (Madison, WI).

Viral vectors also can be used. Representative commercially available viral expression vectors include, but are not limited to, the adenovirus-based Per.C6 system available from Crucell, Inc. (Leiden, The Netherlands), the lentiviral-based pLP1 from ThermoFisher (Carlsbad, CA), and the retroviral vectors pFB-ERV plus pCFB-EGSH from Agilent (Stratagene, La Jolla, CA).

Nucleic acid sequences encoding the inventive amino acid sequences can be provided to a cell on the same vector (i.e., in cis). A unidirectional promoter can be used to control expression of each nucleic acid sequence. In another embodiment, a combination of bidirectional and unidirectional promoters can be used to control expression of multiple nucleic acid sequences. Nucleic acid sequences encoding the inventive amino acid sequences alternatively can be provided to the population of cells on separate vectors (i.e., in trans). Each of the nucleic acid sequences in each of the separate vectors can comprise the same or different expression control sequences. The separate vectors can be provided to cells simultaneously.

The vector(s) comprising the nucleic acid(s) encoding the inventive amino acid sequences can be introduced into a host cell that is capable of expressing the polypeptides encoded thereby, including any suitable prokaryotic or eukaryotic cell. As such, the invention provides an in vitro cell or cell line comprising the inventive vector. The invention also provides an in vitro cell or cell line that expresses the immunoglobulin heavy and/or light chain polypeptides, or expresses the PD-1 binding agent. Preferred host cells are those that can be easily and reliably grown, have reasonably fast growth rates, have well characterized expression systems, and can be transformed or transfected easily and efficiently.

Examples of suitable prokaryotic cells include, but are not limited to, cells from the genera Bacillus (such as Bacillus subtilis and Bacillus brevis), Escherichia (such as E coli), Pseudomonas, Streptomyces, Salmonella, and Erwinia. Particularly useful prokaryotic cells include the various strains of Escherichia coli (e.g., K12, HB101 (ATCC No. 33694), DH5a., DH10, MC1061 (ATCC No. 53338), and CC102).

In some embodiments, the vector is introduced into a eukaryotic cell. Suitable eukaryotic cells are known in the art and include, for example, yeast cells, insect cells, and mammalian cells. Examples of suitable yeast cells include those from the genera Kluyveromyces, Pichia, Rhino-sporidium, Saccharomyces, and Schizosaccharomyces. Preferred yeast cells include, for example, Saccharomyces cerivisae and Pichiapastoris.

Suitable insect cells are described in, for example, Kitts et al., Biotechniques, 14: 810-817 (1993); Lucklow, Curr. Opin. Biotechnol., 4: 564-572 (1993); and Lucklow et al., J. Virol., 67: 4566-4579 (1993). Preferred insect cells include Sf-9 and HI5 (Invitrogen, Carlsbad, CA).

In some embodiments, mammalian cells are utilized in the invention. A number of suitable mammalian host cells are known in the art, and many are available from the American Type Culture Collection (ATCC, Manassas, VA). Examples of suitable mammalian cells include, but are not limited to, Chinese hamster ovary cells (CHO) (e.g., CHO-K1 ATCC No. CCL61), CHO DHFR-cells (e.g., Urlaub et al., Proc. Nall. Acad. Sci. USA, 97: 4216-4220 (1980)), human embryonic kidney (HEK) 293 or 293T cells (e.g., ATCC No. CRL1573), and 3T3 cells (e.g., ATCC No. CCL92). Other suitable mammalian cell lines are the monkey COS-1 (e.g., ATCC No. CRL1650) and COS-7 cell lines (e.g., ATCC No. CRL1651), as well as the CV-1 cell line (e.g., ATCC No. CCL70). Further exemplary mammalian host cells include primate cell lines and rodent cell lines, including the mouse cell line NS0 a derivative of the mouse myeloma line MOPC21 (e.g. Tysabri), and transformed cell lines. Normal diploid cells, cell strains derived from in vitro culture of primary tissue, as well as primary explants, are also suitable. Other suitable mammalian cell lines include, but are not limited to, mouse neuroblastoma N2A cells, HeLa, mouse L-929 cells, and BHK or HaK hamster cell lines, all of which are available from the ATCC. Methods for selecting suitable mammalian host cells and methods for transformation, culture, amplification, screening, and purification of cells are known in the art.

In some embodiments, the mammalian cell is a human cell. For example, the mammalian cell can be a human lymphoid or lymphoid derived cell line, such as a cell line of pre-B lymphocyte origin. Examples of human lymphoid cells lines include, without limitation, RAMOS (e.g., CRL-1596), Daudi (e.g., CCL-213), EB-3 (e.g., CCL-85), Raji cells (e.g., CCL-86), and derivatives thereof.

A nucleic acid sequence encoding the inventive amino acid sequence may be introduced into a cell by any suitable technique, such as by “transfection,” “transformation,” or “transduction.” “Transfection,” “transformation,” or “transduction,” as used herein, refer to the introduction of one or more exogenous polynucleotides into a host cell by using physical or chemical methods. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)). Phage or viral vectors can be introduced into host cells, after growth of infectious particles in suitable packaging cells, many of which are commercially available.

The nucleic acids and cells can be used for any purpose, such as for the manufacture of the CD122 binding agent described herein. In this respect, the invention provides a method of preparing the CD122 binding agent comprising culturing a cell comprising a nucleic acid encoding the heavy and/or light immunoglobulin polypeptides of the CD122 binding agent. Phrased differently, the method comprises expressing a nucleic acid encoding the immunoglobulin heavy and/or light chains of the CD122 binding agent in a cell. It will be appreciated that the immunoglobulin heavy and light chains can be expressed from a single nucleic acid in a given cell, or the immunoglobulin heavy and light chains can be expressed from separate nucleic acids in the same cells. The method can further comprise harvesting and/or purifying the CD122 binding agent from the cell or cell culture media using known techniques.

The following examples further illustrate the invention but, of course, should not be construed as in any way limiting its scope.

### EXAMPLES

The following examples describe particular CD122 binding agent heavy chain polypeptide and light chain polypeptide sequences, according to embodiments of the invention. The antibodies used in these examples are as set forth below.

The antibodies were produced in transient expression in ExpiCHO-S mammalian cells and purified by Protein A affinity chromatography. The antibody sequences for the M10-1275 and M6-p1625 series antibodies are summarized in Tables 1A and 1B, respectively, wherein “H” and “L” chains refer to heavy and light chains. The CDRs of these antibodies as determined according to Kabat numbering are presented in Tables 1C and 1D. The amino acid and nucleic acid sequences for the full heavy and light chains for certain antibodies are summarized in Table IE. The M10-1275 and M6-p1625 represent two classes of antibodies, the members of which were developed through mutagenesis and screening from parental heavy and light chains.

TABLE 1A

| M10-1275 | H Chain Variable Region | L Chain Variable Region |
|---|---|---|
| APE15446 | SEQ ID NO: 24 | SEQ ID NO: 35 |
| APE15276 | SEQ ID NO: 22 | SEQ ID NO: 35 |
| APE15173 | SEQ ID NO: 16 | SEQ ID NO: 35 |
| APE15172 | SEQ ID NO: 16 | SEQ ID NO: 32 |
| APE15171 | SEQ ID NO: 16 | SEQ ID NO: 36 |
| APE15170 | SEQ ID NO: 23 | SEQ ID NO: 35 |
| APE15169 | SEQ ID NO: 23 | SEQ ID NO: 32 |
| APE15168 | SEQ ID NO: 23 | SEQ ID NO: 36 |
| APE15167 | SEQ ID NO: 19 | SEQ ID NO: 28 |
| APE15145 | SEQ ID NO: 17 | SEQ ID NO: 28 |
| APE15144 | SEQ ID NO: 18 | SEQ ID NO: 28 |
| APE15143 | SEQ ID NO: 15 | SEQ ID NO: 28 |
| APE15142 | SEQ ID NO: 16 | SEQ ID NO: 28 |
| APE15141 | SEQ ID NO: 20 | SEQ ID NO: 28 |
| APE15140 | SEQ ID NO: 23 | SEQ ID NO: 28 |
| APE15139 | SEQ ID NO: 21 | SEQ ID NO: 28 |
| APE15138 | SEQ ID NO: 12 | SEQ ID NO: 28 |
| APE15137 | SEQ ID NO: 13 | SEQ ID NO; 28 |
| APE15136 | SEQ ID NO: 14 | SEQ ID NO: 28 |
| APE15135 | SEQ ID NO: 11 | SEQ ID NO: 28 |
| APE15059 | SEQ ID NO: 2 | SEQ ID NO: 28 |
| APE15058 | SEQ ID NO: 2 | SEQ ID NO: 30 |
| APE15057 | SEQ ID NO: 2 | SEQ ID NO: 27 |
| APE15056 | SEQ ID NO: 2 | SEQ ID NO: 35 |
| APE15055 | SEQ ID NO: 2 | SEQ ID NO; 34 |
| APE15054 | SEQ ID NO: 2 | SEQ ID NO: 33 |
| APE15053 | SEQ ID NO: 2 | SEQ ID NO: 32 |
| APE15038 | SEQ ID NO: 10 | SEQ ID NO: 31 |
| APE15037 | SEQ ID NO: 9 | SEQ ID NO: 31 |
| APE15036 | SEQ ID NO: 8 | SEQ ID NO: 31 |
| APE15035 | SEQ ID NO: 4 | SEQ ID NO: 31 |
| APE15034 | SEQ ID NO: 3 | SEQ ID NO: 31 |
| APE15033 | SEQ ID NO: 5 | SEQ ID NO: 31 |
| APE15032 | SEQ ID NO: 7 | SEQ ID NO: 31 |
| APE15031 | SEQ ID NO: 6 | SEQ ID NO: 31 |
| APE14970 | SEQ ID NO: 2 | SEQ ID NO: 31 |
| APE14969 | SEQ ID NO: 2 | SEQ ID NO: 29 |
| APE14971 | SEQ ID NO: 2 | SEQ ID NO: 26 |
| APE12705 | SEQ ID NO: 1 | SEQ ID NO: 25 |

TABLE 1B

| M6-p1625 | H Chain Variable Region | L Chain Variable Region |
|---|---|---|
| APE15428 | SEQ ID NO: 69 | SEQ ID NO: 77 |
| APE15295 | SEQ ID NO: 68 | SEQ ID NO: 77 |
| APE15371 | SEQ ID NO: 45 | SEQ ID NO: 77 |
| APE15370 | SEQ ID NO: 46 | SEQ ID NO: 77 |
| APE15369 | SEQ ID NO: 63 | SEQ ID NO: 77 |
| APE15368 | SEQ ID NO: 64 | SEQ ID NO: 77 |
| APE15300 | SEQ ID NO: 67 | SEQ ID NO: 77 |
| APE15299 | SEQ ID NO: 59 | SEQ ID NO: 77 |
| APE15298 | SEQ ID NO: 53 | SEQ ID NO: 77 |
| APE15297 | SEQ ID NO: 66 | SEQ ID NO: 77 |
| APE15296 | SEQ ID NO: 54 | SEQ ID NO: 77 |
| APE15295 | SEQ ID NO: 68 | SEQ ID NO: 77 |
| APE15293 | SEQ ID NO: 55 | SEQ ID NO: 77 |
| APE15292 | SEQ ID NO: 47 | SEQ ID NO: 77 |
| APE15291 | SEQ ID NO: 56 | SEQ ID NO: 77 |
| APE15290 | SEQ ID NO: 60 | SEQ ID NO: 77 |
| APE15288 | SEQ ID NO: 61 | SEQ ID NO: 77 |
| APE15287 | SEQ ID NO: 48 | SEQ ID NO: 77 |
| APE15286 | SEQ ID NO: 49 | SEQ ID NO: 77 |
| APE15285 | SEQ ID NO: 65 | SEQ ID NO: 77 |
| APE15284 | SEQ ID NO: 50 | SEQ ID NO: 77 |
| APE15283 | SEQ ID NO: 58 | SEQ ID NO: 77 |
| APE15280 | SEQ ID NO: 43 | SEQ ID NO: 77 |
| APE15279 | SEQ ID NO: 57 | SEQ ID NO: 77 |
| APE15278 | SEQ ID NO: 62 | SEQ ID NO: 77 |
| APE15277 | SEQ ID NO: 51 | SEQ ID NO: 77 |
| APE15148 | SEQ ID NO: 40 | SEQ ID NO: 77 |
| APE15093 | SEQ ID NO: 39 | SEQ ID NO: 72 |
| APE15092 | SEQ ID NO: 40 | SEQ ID NO: 72 |
| APE15091 | SEQ ID NO: 41 | SEQ ID NO: 72 |
| APE15090 | SEQ ID NO: 42 | SEQ ID NO: 72 |
| APE15089 | SEQ ID NO: 38 | SEQ ID NO: 73 |
| APE15088 | SEQ ID NO: 38 | SEQ ID NO: 74 |
| APE15087 | SEQ ID NO: 38 | SEQ ID NO: 75 |
| APE15086 | SEQ ID NO: 38 | SEQ ID NO: 76 |
| APE15085 | SEQ ID NO: 38 | SEQ ID NO: 77 |
| APE14950 | SEQ ID NO: 38 | SEQ ID NO: 72 |
| APE14949 | SEQ ID NO: 44 | SEQ ID NO: 71 |
| APE14948 | SEQ ID NO: 44 | SEQ ID NO: 72 |
| APE14947 | SEQ ID NO: 52 | SEQ ID NO: 71 |
| APE14946 | SEQ ID NO: 52 | SEQ ID NO: 72 |
| APE14951 | SEQ ID NO: 38 | SEQ ID NO: 71 |
| APE12383 | SEQ ID NO: 37 | SEQ ID NO: 70 |

TABLE 1C

| | CDR Sequences (SEQ ID NO) | | | | | |
|---|---|---|---|---|---|---|
| M10-1275 | CDRH1 | CDRH2 | CDRH3 | CDRL1 | CDRL2 | CDRL3 |
| APE15446 | 117 | 127 | 136 | 138 | 139 | 144 |
| APE15276 | 117 | 127 | 137 | 138 | 139 | 144 |
| APE15173 | 124 | 127 | 136 | 138 | 139 | 144 |
| APE15172 | 124 | 127 | 136 | 138 | 139 | 141 |
| APE15171 | 124 | 127 | 136 | 138 | 139 | 140 |
| APE15170 | 117 | 127 | 136 | 138 | 139 | 144 |
| APE15169 | 117 | 127 | 136 | 138 | 139 | 141 |
| APE15168 | 117 | 127 | 136 | 138 | 139 | 140 |
| APE15167 | 123 | 130 | 136 | 138 | 139 | 145 |
| APE15145 | 121 | 127 | 136 | 138 | 139 | 145 |
| APE15144 | 125 | 127 | 136 | 138 | 139 | 145 |
| APE15143 | 122 | 127 | 136 | 138 | 139 | 145 |
| APE15142 | 124 | 127 | 136 | 138 | 139 | 145 |
| APE15141 | 123 | 127 | 136 | 138 | 139 | 145 |
| APE15140 | 117 | 127 | 136 | 138 | 139 | 145 |
| APE15139 | 117 | 130 | 136 | 138 | 139 | 145 |
| APE15138 | 121 | 129 | 136 | 138 | 139 | 145 |
| APE15137 | 122 | 129 | 136 | 138 | 139 | 145 |
| APE15136 | 123 | 129 | 136 | 138 | 139 | 145 |
| APE15135 | 126 | 128 | 136 | 138 | 139 | 145 |
| APE15059 | 126 | 129 | 136 | 138 | 139 | 145 |
| APE15058 | 126 | 129 | 136 | 138 | 139 | 145 |
| APE15057 | 126 | 129 | 136 | 138 | 139 | 145 |
| APE15056 | 126 | 129 | 136 | 138 | 139 | 144 |
| APE15055 | 126 | 129 | 136 | 138 | 139 | 143 |
| APE15054 | 126 | 129 | 136 | 138 | 139 | 142 |
| APE15053 | 126 | 129 | 136 | 138 | 139 | 141 |
| APE15038 | 120 | 129 | 136 | 138 | 139 | 145 |
| APE15037 | 119 | 129 | 136 | 138 | 139 | 145 |
| APE15036 | 118 | 129 | 136 | 138 | 139 | 145 |
| APE15035 | 126 | 131 | 136 | 138 | 139 | 145 |
| APE15034 | 126 | 132 | 136 | 138 | 139 | 145 |
| APE15033 | 126 | 133 | 136 | 138 | 139 | 145 |
| APE15032 | 126 | 135 | 136 | 138 | 139 | 145 |
| APE15031 | 126 | 134 | 136 | 138 | 139 | 145 |
| APE14970 | 126 | 129 | 136 | 138 | 139 | 145 |
| APE14969 | 126 | 129 | 136 | 138 | 139 | 145 |
| APE14971 | 126 | 129 | 136 | 138 | 139 | 145 |
| APE12705 | 126 | 129 | 136 | 138 | 139 | 145 |

TABLE 1D

| | CDR Sequences (SEQ ID NO) | | | | | |
|---|---|---|---|---|---|---|
| M6-1625 | CRH1 | CDRH2 | CDRH3 | CDRL1 | CDRL2 | CDRL3 |
| APE15428 | 78 | 94 | 98 | 99 | 103 | 105 |
| APE15295 | 78 | 94 | 98 | 99 | 103 | 105 |
| APE15371 | 80 | 94 | 98 | 99 | 103 | 105 |
| APE15370 | 80 | 94 | 98 | 99 | 103 | 105 |
| APE15369 | 78 | 94 | 98 | 99 | 103 | 105 |
| APE15368 | 78 | 94 | 98 | 99 | 103 | 105 |
| APE15300 | 93 | 94 | 98 | 99 | 103 | 105 |
| APE15299 | 89 | 94 | 98 | 99 | 103 | 105 |
| APE15298 | 80 | 96 | 98 | 99 | 103 | 105 |
| APE15297 | 92 | 94 | 98 | 99 | 103 | 105 |
| APE15296 | 85 | 94 | 98 | 99 | 103 | 105 |
| APE15295 | 78 | 94 | 98 | 99 | 103 | 105 |
| APE15293 | 86 | 94 | 98 | 99 | 103 | 105 |
| APE15292 | 83 | 94 | 98 | 99 | 103 | 105 |
| APE15291 | 87 | 94 | 98 | 99 | 103 | 105 |
| APE15290 | 84 | 94 | 98 | 99 | 103 | 105 |
| APE15288 | 90 | 94 | 98 | 99 | 103 | 105 |
| APE15287 | 80 | 94 | 98 | 99 | 103 | 105 |
| APE15286 | 80 | 94 | 98 | 99 | 103 | 105 |
| APE15285 | 91 | 94 | 98 | 99 | 103 | 105 |
| APE15284 | 80 | 94 | 98 | 99 | 103 | 105 |
| APE15283 | 84 | 94 | 98 | 99 | 103 | 105 |
| APE15280 | 82 | 94 | 98 | 99 | 103 | 105 |
| APE15279 | 88 | 94 | 98 | 99 | 103 | 105 |
| APE15278 | 79 | 97 | 98 | 99 | 103 | 105 |
| APE15277 | 84 | 94 | 98 | 99 | 103 | 105 |
| APE15148 | 80 | 94 | 98 | 99 | 103 | 105 |
| APE15093 | 79 | 94 | 98 | 99 | 103 | 107 |
| APE15092 | 80 | 94 | 98 | 99 | 103 | 107 |
| APE15091 | 81 | 94 | 98 | 99 | 103 | 107 |
| APE15090 | 79 | 95 | 98 | 99 | 103 | 107 |
| APE15089 | 79 | 94 | 98 | 100 | 103 | 107 |
| APE15088 | 79 | 94 | 98 | 101 | 103 | 107 |
| APE15087 | 79 | 94 | 98 | 102 | 103 | 107 |
| APE15086 | 79 | 94 | 98 | 99 | 103 | 106 |
| APE15085 | 79 | 94 | 98 | 99 | 103 | 105 |
| APE14950 | 79 | 94 | 98 | 99 | 103 | 107 |
| APE14949 | 79 | 94 | 98 | 99 | 104 | 107 |
| APE14948 | 79 | 94 | 98 | 99 | 103 | 107 |
| APE14947 | 79 | 94 | 98 | 99 | 104 | 107 |
| APE14946 | 79 | 94 | 98 | 99 | 103 | 107 |
| APE14951 | 79 | 94 | 98 | 99 | 104 | 107 |
| APE12383 | 79 | 94 | 98 | 99 | 104 | 107 |

TABLE 1E

| | | | | |
|---|---|---|---|---|
| | H Chain | H Chain | L Chain | L Chain |
| Antibody | Amino Acid | Nucleic Acid | Amino Acid | Nucleic Acid |

M10-1275

| | | | | |
|---|---|---|---|---|
| APE15446 | 152 | 154 | 153 | 155 |
| APE15170 | 157 | 156 | 153 | 155 |
| APE15142 | 159 | 158 | 161 | 160 |

M6-P1625

| | | | | |
|---|---|---|---|---|
| APE15428 | 115 | 162 | 116 | 163 |
| APE15295 | 165 | 164 | 116 | 163 |

### Example 1

This example demonstrates the binding kinetics (affinities) of the antibodies disclosed herein.

Surface plasmon resonance (SPR) was used to determine the binding affinities. The M6-1625 and M10-1275 antibodies were captured onto a Cytiva Protein A Chip for 60 seconds at 0.5 μg/mL for 60 seconds. The antibodies were exposed to recombinant human LL-2Rβ or cynomolgus LL-2Rβ (corresponding to residues Ala1-Thr215 of the extracellular domains excluding the signal peptide) at typical concentrations ranging from 0.4 nM to 11 nM with typical 300 second associations and 1200 second dissociations using a BiacoreS200 instrument. Sensorgrams were fit globally using a 1:1 binding model in the Biacore T200 Evaluation Software to calculate on- and off-rates and K_{D} values. A summary of K_{D} measurements for the CD122 binding agents disclosed herein by SPR is set forth in Tables 2A and 2B.

TABLE 2A
M6-p1625 Binding Affinity Data

| Antibody | Human IL-2Rβ K_{D} (SPR) | | Cynomolgus IL-2Rβ K_{D} (SPR) | |
|---|---|---|---|---|
| APE15428 | 56.7 | pM | 76.5 | pM |
| APE15295 | 55.7 | pM | 70.7 | pM |
| APE15371 | 91.7 | pM | 82.1 | pM |
| APE15370 | 110 | pM | 98.2 | pM |
| APE15369 | 67.4 | pM | 63.4 | pM |
| APE15368 | 86.7 | pM | 78.3 | pM |
| APE15300 | 1.41 | nM | 1.16 | nM |
| APE15299 | 348 | pM | 390 | pM |
| APE15298 | 98.1 | pM | 132 | pM |
| APE15297 | 157 | pM | 153 | pM |
| APE15296 | 851 | pM | 825 | pM |
| APE15295 | 55.7 | pM | 70.7 | pM |
| APE15293 | 351 | pM | 359 | pM |
| APE15292 | 376 | pM | 437 | pM |
| APE15291 | 1.98 | nM | 2.68 | nM |
| APE15290 | 261 | pM | 305 | pM |
| APE15288 | 172 | pM | 199 | pM |
| APE15287 | 169 | pM | 186 | pM |
| APE15286 | 87.3 | pM | 104 | pM |
| APE15285 | 310 | pM | 327 | pM |
| APE15284 | 56.6 | pM | 70.5 | pM |
| APE15283 | 312 | pM | 318 | pM |
| APE15280 | 161 | pM | 202 | pM |
| APE15279 | 522 | pM | 533 | pM |
| APE15278 | 334 | pM | 488 | pM |
| APE15277 | 229 | pM | 244 | pM |
| APE15148 | 94.5 | pM | 71.5 | pM |
| APE15093 | 324/238 | pM | 189 | pM |
| APE15092 | 234/162 | pM | 126 | pM |
| APE15091 | 577/442 | pM | 345 | pM |
| APE15090 | 657 | pM | 440 | pM |
| APE15089 | 1.04 | nM | 9.39 | nM |
| APE15088 | 16.4 | nM | 16.7 | nM |
| APE15087 | 4.31 | nM | 3.56 | nM |
| APE15086 | 305 | pM | 250 | pM |
| APE15085 | 158 | pM | 154 | pM |
| APE14950 | 252 | pM | 192 | pM |
| APE14949 | 1.5 | nM | 1 | nM |
| APE14948 | 358 | pM | 266 | pM |
| APE14947 | 1.46 | nM | 977 | pM |
| APE14946 | 357 | pM | 255 | pM |
| APE14951 | 804 | pM | 584 | pM |
| APE12383 | 340 | pM | 260 | pM |

TABLE 2B
M10-1275 Binding Affinity Data

| Antibody | Human IL-2Rβ K_{D} (SPR) | | Cynomolgus IL-2Rβ K_{D} (SPR) | |
|---|---|---|---|---|
| APE15446 | 10.7 | pM | 4.15 | pM |
| APE15276 | 17.8 | pM | 5.99 | pM |
| APE15173 | 18.4 | pM, | 5.09 | pM, |
| APE15172 | 116 | pM, | 27.1 | pM, |
| APE15171 | 276 | pM | 52.2 | pM |
| APE15170 | 12.8 | pM, | 4.87 | pM, |
| APE15169 | 99.5 | pM, | 14.4 | pM, |
| APE15168 | 324 | pM | 37.9 | pM |
| APE15167 | 45 | pM | 6.55 | pM |
| APE15145 | 29.6 | pM | 10.8 | pM |
| APE15144 | 29.7 | pM | 11.6 | pM |
| APE15143 | 25.1 | pM | 9.72 | pM |
| APE15142 | 22.2 | pM | 10.5 | pM |
| APE15141 | 22.0 | pM | 10.3 | pM |
| APE15140 | 21.7 | pM | 10.2 | pM |
| APE15139 | 35.2 | pM | 9.23 | pM |
| APE15138 | 43.6 | pM | 16.3 | pM |
| APE15137 | 51.8 | pM | 17.4 | pM |
| APE15136 | 48.1 | pM | 11.4 | pM |
| APE15135 | 28.2 | pM | 10.5 | pM |
| APE15059 | 69.7 | pM | 15.9 | pM |
| APE15058 | 154 | pM | 22.3 | pM |
| APE15057 | 144 | pM | 24 | pM |
| APE15056 | 122 | pM | 19.8 | pM |
| APE15055 | 369 | pM | 83.8 | pM |
| APE15054 | 429 | pM | 108 | pM |
| APE15053 | 91.5 | pM | 39.9 | pM |
| APE15038 | 120 | pM | 18.5 | pM |
| APE15037 | 124 | pM | 17.1 | pM |
| APE15036 | 165 | pM | 19.5 | pM |
| APE15035 | 147 | pM | 19.4 | pM |
| APE15034 | 190 | pM | 22.2 | pM |
| APE15033 | 106 | pM | 12.8 | pM |
| APE15032 | 73.8 | pM | 12.4 | pM |
| APE15031 | 108 | pM | 18.9 | pM |
| APE14970 | 74 | pM | 14 | pM |
| APE14969 | 187 | pM | 18 | pM |
| APE14971 | 278 | pM | 26 | pM |
| APE12705 | 80 | pM | 20 | pM |

### Example 2

This Example demonstrates that the inventive anti-CD122 chimeric antibodies bind in a dose-dependent manner to human or cynomolgus IL-2Rβγ expressed on CHO-K1 cells.

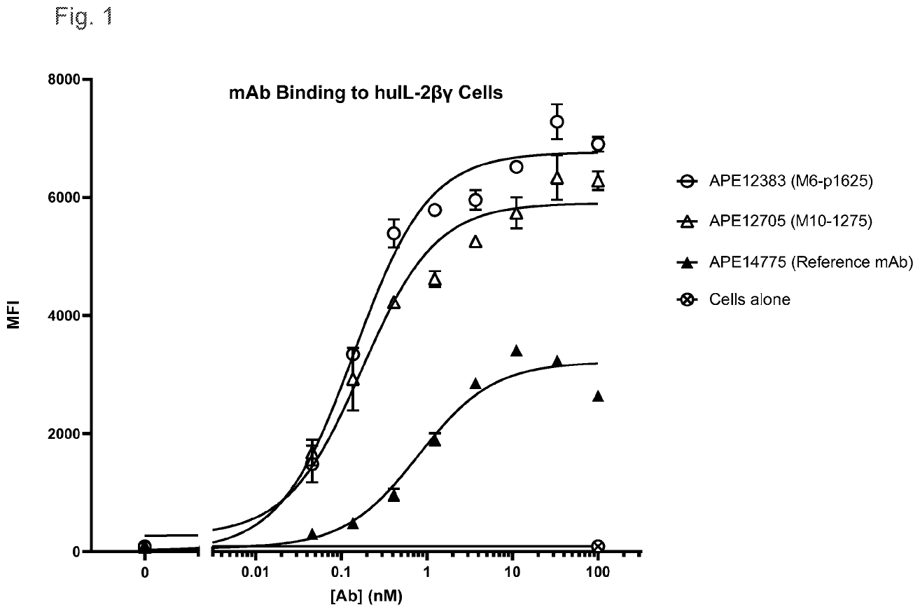

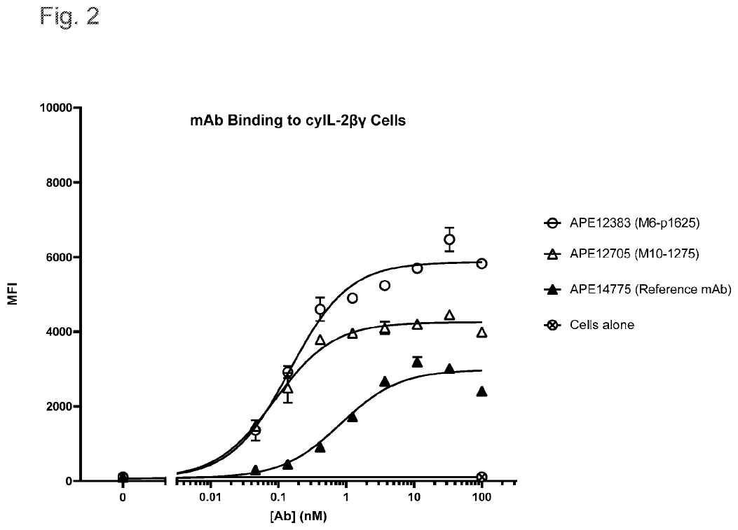

CHO-K1 cells stably expressing full-length human or cynomolgus IL-2Rβ and IL-2Rγ cultured in the log phase of growth were harvested with Accutase solution, resuspended in FACS buffer, and plated at 100,000 cells/well in a 96-well plate. The concentrations of antibodies indicated in FIGS. 1 and 2 starting at 100 nM and diluted 1:3 were incubated with the cells for 45 minutes on a plate shaker at 4° C. After washing, the cells were incubated with goat-anti-human kappa DyL650 secondary antibody for 20 minutes on a plate shaker at 4° C. Cells were washed again and analyzed on a FACSArray flow cytometer. Data were analyzed using Novoexpress and GraphPad Prism software. Results are shown in FIGS. 1 and 2. Table 3 lists anti-CD122 chimeric antibody binding characteristics: affinity to human and cyno CD122 protein (K_{D}, measured by SPR assay), ability to block IL-2 (SPR assay), and binding EC50 to cell-expressed human and cyno CD122/CD132 (cell-based receptor binding assay).

TABLE 3

| | | Human | Binding | | h | c |
|---|---|---|---|---|---|---|
| | | IL- | to | | IL- | IL- |
| | | Binding | Cyno | Block- | 2RB | 2RB |
| | De- | to | IL- | ing | cell | cell |
| APE | scrip- | 2RB | 2RB | (IL-2 | EC50 | EC50 |
| Number | tion | (~KD) | (~KD) | down) | (nM) | (nM) |
| APE | M6- | 340 pM | 260 pM | Blocker | 0.1424 | 0.1467 |
| 12383.03 | p1625 | | | | | |
| APE | M10- | 80 pM | 20 pM | Partial | 0.1736 | 0.08761 |
| 12705.02 | 1275 | | | blocker | | |
| | | | | (show- | | |
| | | | | ing | | |
| | | | | in- | | |
| | | | | com- | | |
| | | | | plete | | |
| | | | | by | | |
| | | | | SPR) | | |

### Example 3

This Example demonstrates that the anti-CD122 antibodies of the present invention are capable of inhibiting IL-2-induced or IL-15-induced pSTAT5 luciferase activity in HEK cells expressing human IL-2βγ.

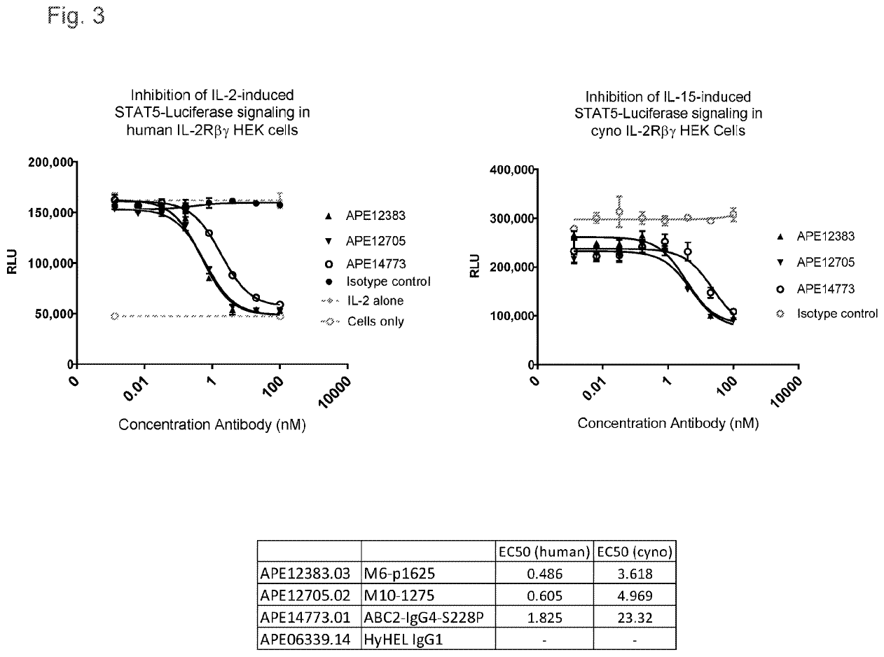

HEK293 cells stably transfected with IL-2Rβ, IL-2Rγ, and STAT5-luciferase reporter were plated at 50,000 cells per well in a 96-well plate. Chimeric antibodies or JAK inhibitors were titrated at the concentrations indicated in FIG. 3, starting at 100 nM, and added to the cells for 30 minutes. Recombinant IL-2 or IL-15 was then added to the cells for an additional 3.5 hours. SteadyGlo substrate was added for 10 minutes and the plates were read on the GloMax machine. Data were analyzed using GraphPad Prism software. Results are shown in FIG. 3.

### Example 4

This Example demonstrates that the anti-CD122 IgG4 antibodies are able to inhibit IL-2- or IL-15-induced proliferation of PHA-stimulated primary human T cell blasts.

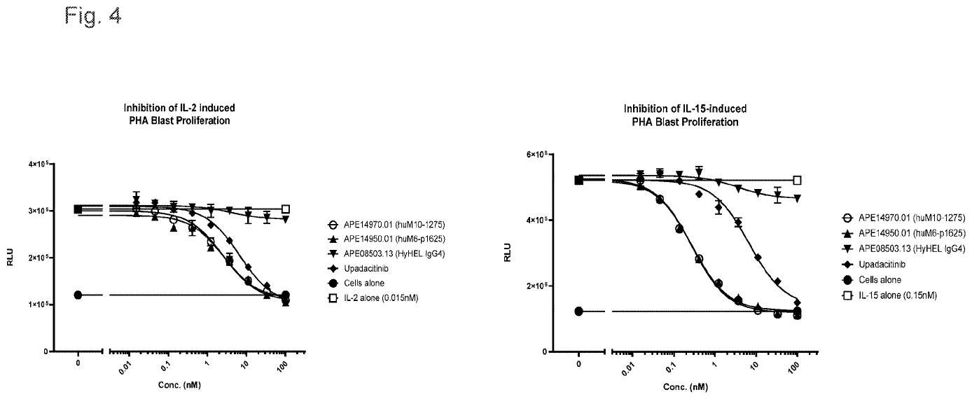

Primary human PBMCs were isolated from whole blood donors, and stimulated in cuture for 3 days with 5 μg/mL PHA in a T75 flask. Cells were washed, counted, and plated at 100,000 cells/well in a 96-well plate. Antibodies were titrated at the concentrations indicated in FIG. 4, starting at 100 nM with 1:3 dilutions, and added to the cells. Recombinant IL-2 (15 pM) or IL-15 (150 pM) were added to the cells. Cells were cultured for an additional three days. SteadyGlo substrate was added for 10 minutes and the plates were read on the GloMax machine. Data were analyzed using Graphpad Prism software.

Results are shown in FIG. 4. Both M10-1275 and M6-1625 were better than upadacitinib at blocking IL-2 and IL-15.

### Example 5

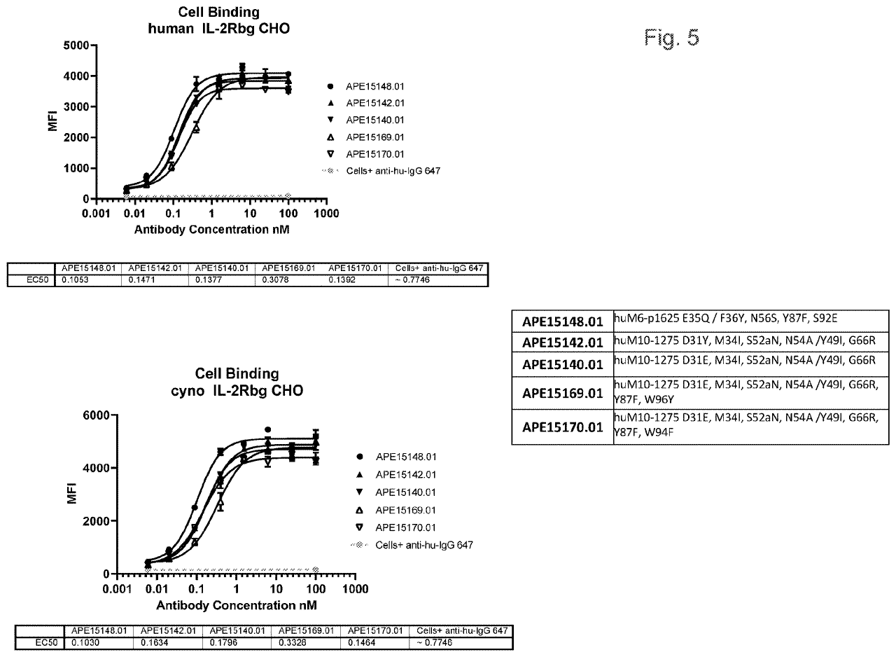

This Example demonstrates that the anti-CD122 IgG4 antibodies bind in a dose-dependent manner to human IL-2Rβγ expressed on CHO-K1 cells. CHO-K1 cells stably expressing either full-length human or cyno IL-2Rβ and IL-2Rγ cultured in the log phase of growth were harvested with Accutase solution, resuspended in FACS buffer, and plated at 100,000 cells/well in a 96 well plate. The concentrations indicated in FIG. 5 of antibodies starting at 100 nM and diluted 1:3 were incubated with the cells for 45 minutes on a plate shaker at4° C. After washing, cells were incubated with goat anti-human kappa DyL650 secondary antibody for 20 minutes on a plate shaker at4° C. Cells were washed again and analyzed on a FACSArray flow cytometer. Data were analyzed using Novoexpress and GraphPad Prism software.

Results are shown in FIG. 5. All antibodies show human and cyno binding with EC50s in the range of 0.3-0.1 nM range.

### Example 6

This Example demonstrates that humanized anti-CD122 IgG4 antibodies inhibit IL-2- or IL-15-induced proliferation of purified primary human NK cells.

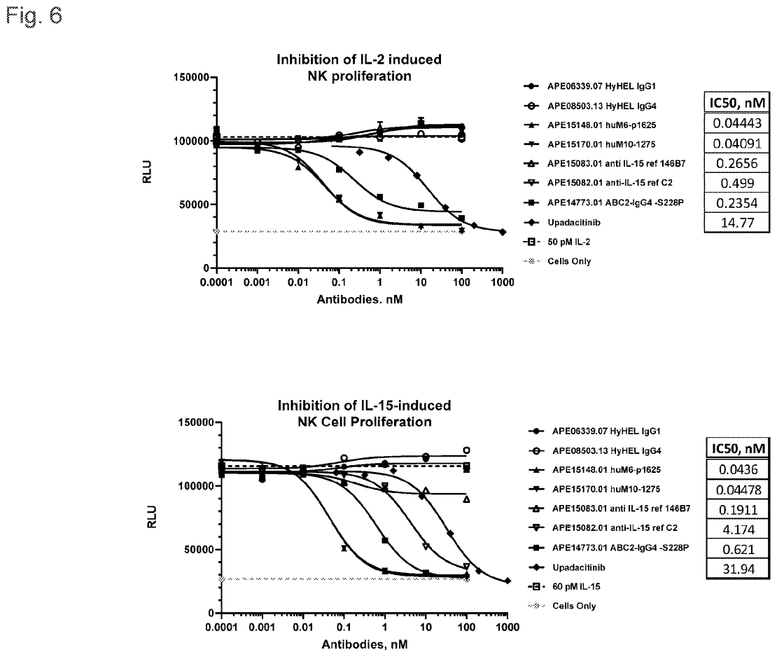

Primary human PBMCs were isolated from whole blood donors, and untouched NK cells were isolated using a Miltenyi bead kit. 100,000 cells were plated in a 96 well plate. Antibodies were titrated at the indicated concentrations, starting at 100 nM with 1:10 dilutions, and added to the cells. Recombinant IL-2 (50 pM) or recombinant IL-15 (60 pM) were added to the cells. Cells were cultured for 5 days. Finally, SteadyGlo substrate was added for 10 minutes and the plates read on the GloMax machine. Data were analyzed using Graphpad Prism software. Results are shown in FIG. 6.

### Example 7

This Example demonstrates that humanized anti-CD122 IgG4 antibodies inhibit IL-2- or IL-15-induced proliferation and survival of primary human NK cells, gated within a PBMC culture.

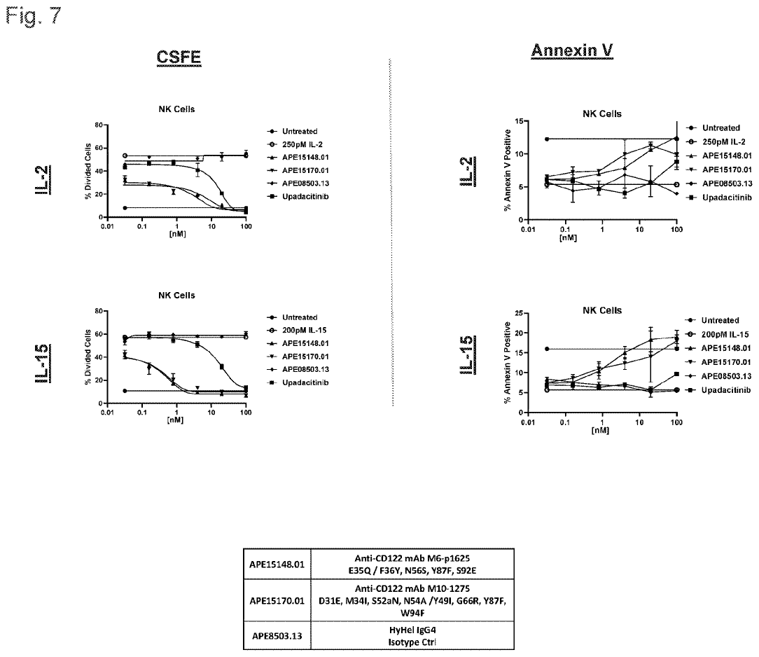

Primary human PBMCs were isolated from whole blood donors, labeled with CFSE, and plated at 250,000 cells/well in a 96 well plate. Antibodies were titrated at the concentrations indicated in FIG. 7, starting at 100 nM and diluting 1:3, and added to the cells. Recombinant IL-2 (250 pM) or recombinant IL-15 (200 pM) were added to the cells. Cells were incubated for 5 days. Cells were stained with CD56 and CD3 to gate on the NK cell population, and with Annexin V as a measure of apoptotic cells. Cells were analyzed on a flow cytometer and data were analyzed using Novoexpress and GraphPad Prism software. Proliferation is indicated by the % of cells with diluted CFSE peaks (beyond the unstimulated fraction), and apoptosis is indicated by annexin V staining, as shown in FIG. 7. Both of the tested antibodies effectively inhibited IL-2 or IL-15 induced proliferation, as measured by CFSE.

### Example 8

This Example demonstrates that humanized anti-CD122 IgG4 antibodies inhibit IL-2- or IL-15 binding to HEK cells expressing human IL-2βγ.

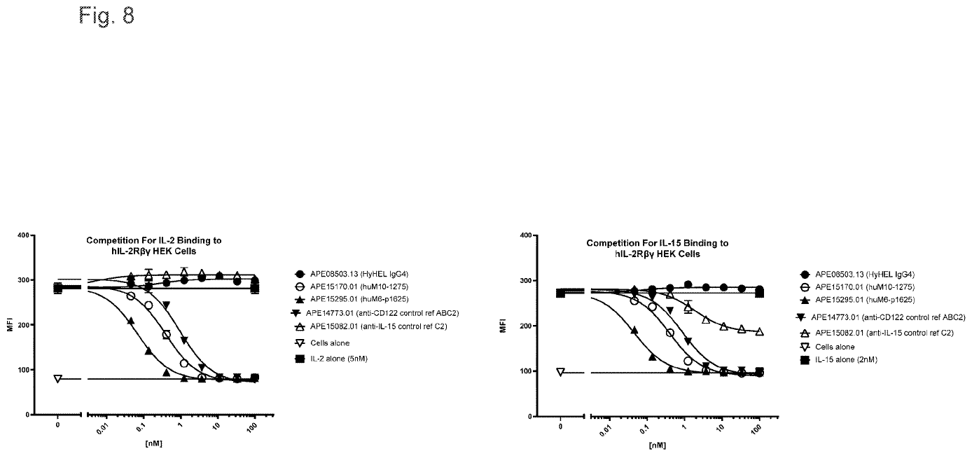

HEK293 cells stably transfected with human IL-2Rβ and IL-2Rγ were plated at 100,000 cells/well in a 96 well plate. Antibodies were titrated at the concentrations indicated in FIG. 8 starting at 100 nM and diluted 1:3, and added to the cells. Recombinant IL-2 (5 nM) or recombinant IL-15 (2 nM) were also added to the cells. Cells were incubated for 30 minutes on a plate shaker at 4° C. Cells were washed and then analyzed on a flow cytometer, and data were analyzed using Novoexpress and GraphPad Prism software. Results are shown in FIG. 8.

### Example 9

This Example demonstrates that humanized anti-CD122 IgG4 antibodies inhibit IL-15 or IL-15/IL1-5RA induced proliferation of primary human T cell blasts.

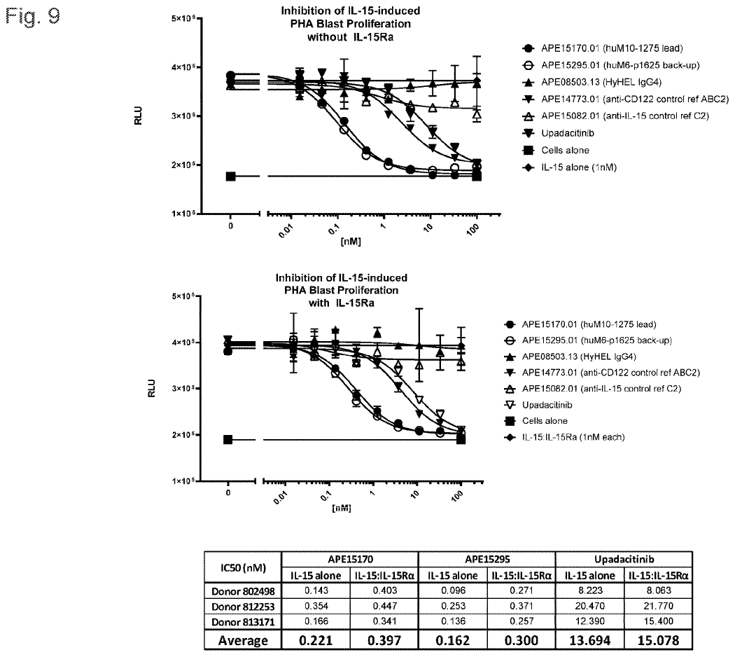

Primary human PBMCs were isolated from whole blood donors, and stimulated in culture for 3 days with 5 ug/mL PHA in a T75 flask. Cells were washed, counted, and plated at 100,000 cells/well in a 96 well plate. Antibodies were titrated at the concentrations indicated in FIG. 9, starting at 100 nM with 1:3 dilutions, and added to the cells. Recombinant IL-15 alone (1 nM) or recombinant IL-15+IL-15Ra (1 nM each) were added to the cells. Cells were cultured for an additional three days. Finally, SteadyGlo substrate was added for 10 minutes and the plates read on the GloMax machine. Data were analyzed using Graphpad Prism software. Results are shown in FIG. 9.

### Example 10

This Example demonstrates that humanized anti-CD122 IgG4 antibodies inhibit IL-15 or IL-15/IL1-5RA induced proliferation of primary human NK cells.

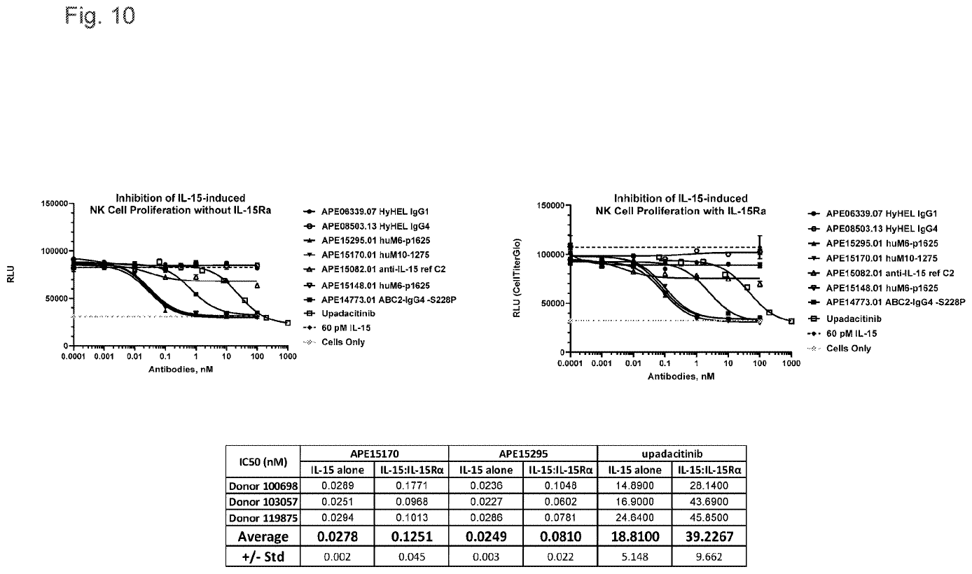

Primary human PBMCs were isolated from whole blood donors, and untouched NK cells were isolated using a Miltenyi bead kit. 100,000 cells were plated in a 96 well plate. Antibodies were titrated at the concentrations indicated in FIG. 10, starting at 100 nM with 1:10 dilutions, and added to the cells. Recombinant IL-15 (60 pM) or recombinant IL-15:IL15RA (60 pM each) were added to the cells. Cells were cultured for 5 days. Finally, SteadyGlo substrate was added for 10 minutes and the plates read on the GloMax machine. Data were analyzed using Graphpad Prism software. Results are shown in FIG. 10

### Example 11

This Example demonstrates that humanized anti-CD122 IgG1 antibodies inhibit IL-2- or IL-15-induced proliferation of primary human NK cells.

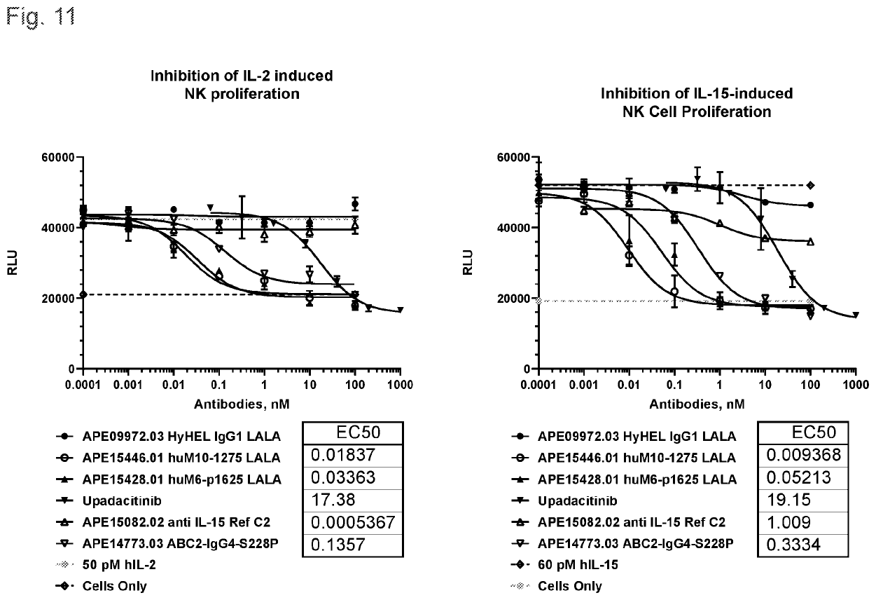

Primary human PBMCs were isolated from whole blood donors, and untouched NK cells were isolated using a Miltenyi bead kit. 100,000 cells were plated in a 96 well plate. Antibodies were titrated at the concentrations indicated in FIG. 11, starting at 100 nM with 1:10 dilutions, and added to the cells. Recombinant IL-2 (50 pM) or recombinant IL-15 (60 pM) were added to the cells. Cells were cultured for 5 days. Finally, SteadyGlo substrate was added for 10 minutes and the plates read on the GloMax machine. Data were analyzed using Graphpad Prism software. Results are shown in FIG. 11

### Example 12

This Example demonstrates that humanized anti-CD122 IgG1 antibodies inhibit IL-2- or IL-15-induced proliferation of primary human pan T cells.

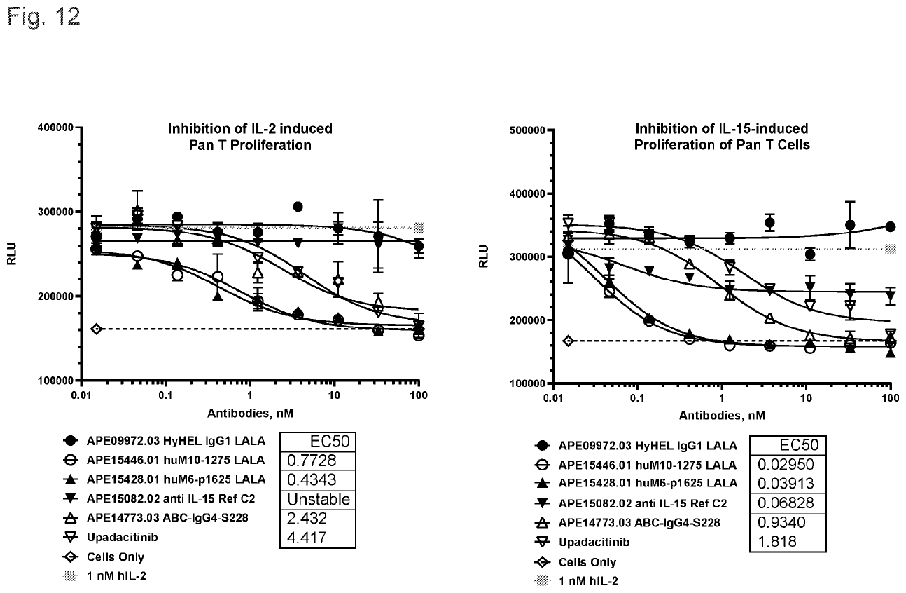

Primary human PBMCs were isolated from whole blood donors, and untouched pan T cells were isolated using a Miltenyi bead kit. 100,000 cells were plated in a 96 well plate. Antibodies were titrated at the concentrations indicated in FIG. 12, starting at 100 nM with 1:3 dilutions, and added to the cells. Recombinant IL-2 (1 nM) or recombinant IL-15 were added to the cells. Cells were cultured for 7 days. Finally, SteadyGlo substrate was added for 10 minutes and the plates read on the GloMax machine. Data were analyzed using Graphpad Prism software. Results are shown in FIG. 12.

### Example 13

This Example demonstrates that humanized anti-CD122 antibodies show efficacy in vivo in a xenogenic NSG-Tg(huIL-15)/Hu-PBMC Graft v. Host Disease (GvHD) model.

A xenogenic NSG-Tg(huIL-15)/Hu-PBMC GvHD model was performed. NOD-scidIL2rγ^{null} (NSG) human IL-15 transgenic mice were irradiated with 1 Gy followed by intravenous injection of human PBMCs in each mouse. Antibodies (APE15170-IgG4P, APE15295-IgG4P, CTLA4-Ig (positive control), and APE15170-IgG4 in combination with CTLA4-Ig) were dosed intraperitoneally. APE15170-IgG4P, APE15295-IgG4P, and APE15170-IgG4P were dosed two times per week for 4 weeks at 10 mg/kg, and CTLA4-Ig was dosed three times per week at 75 μg, both starting the day following PBMC injection. A control IgG4 isotype was also dosed two times per week.

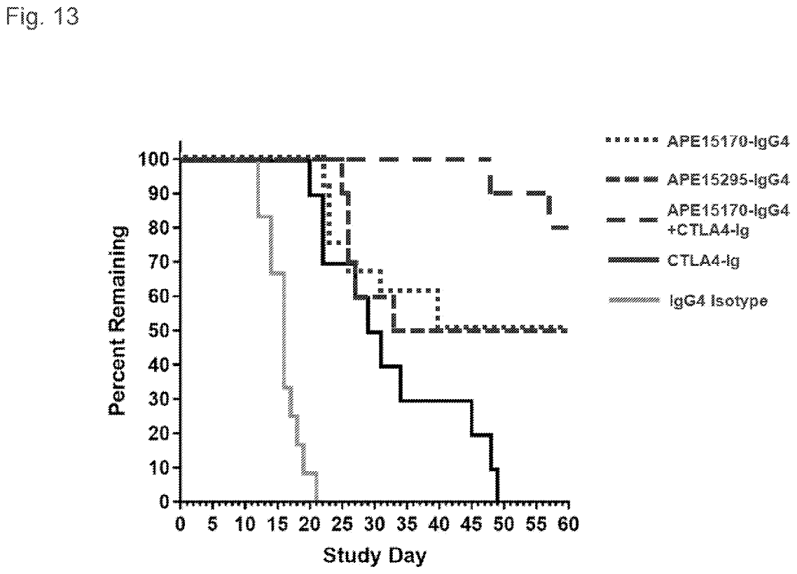

The anti-CD122 antagonist antibodies disclosed herein show significant improvement vs. isotype control and CTLA4-Ig alone in prevention of death. APE15170-IgG4P in combination with CTLA4-Ig showed even better improvement than APE15170-IgG4P or APE15295-IgG4P alone. Results are shown in FIG. 13.

### Example 14

This Example demonstrates that humanized anti-CD122 antibodies show efficacy in vivo in a xenogeneic NSG(huIL-15Tg)/Hu-PBMC Graft vs. Host Disease (GvHD) model.

A xenogeneic NSG(huIL-15Tg)/Hu-PBMC GvHD model testing the efficacy of the anti-CD122 antibody disclosed herein was performed at The Jackson laboratory JAX® In Vivo Pharmacology Services (Sacramento, CA). NOD-scidIL2rγ^{null} (NSG) human IL-15 transgenic mice were irradiated with 1 Gy followed by intravenous injection of human PBMCs in each mouse. APE15428 (IgG1-LALA) was dosed intraperitoneally at 10 mg/kg, 3 mg/kg, or 1 mg/kg twice weekly for 4 weeks starting the day following PBMC injection. Disease was monitored three times weekly by body weight loss, death, and GvHD scores measuring: weight loss, activity, fur texture, paleness, and posture. Animals exhibiting more than 10% body weight loss were disease monitored daily, and animals exhibiting more than 20% body weight loss from starting weight were euthanized.

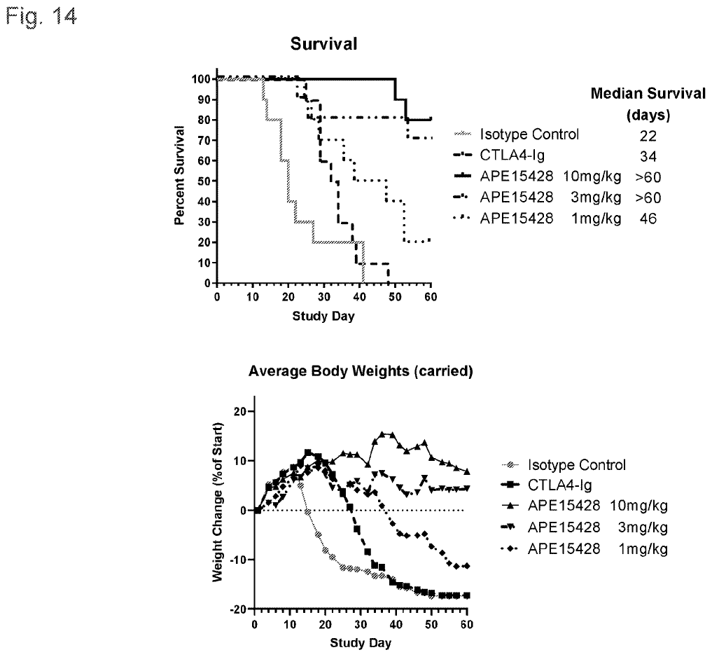

The anti-CD122 antagonist antibody disclosed herein showed statistically significant efficacy vs. isotype control in median time to survival. Individual animals' percent of start body weight are shown when dosed with isotype control, anti-CD122 antibody APE15428 (IgG1 LALA), and CTLA-4-Ig (positive control), respectively. Results are shown in FIG. 14.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A CD122 binding agent comprising an immunoglobulin heavy chain variable region and an immunoglobulin light chain variable region, wherein
(a) the immunoglobulin heavy chain variable region comprises:
a CDR1 comprising SEQ ID NO: 108;
a CDR2 comprising SEQ ID NO: 109; and
a CDR3 comprising SEQ ID NO: 98;
and the immunoglobulin light chain variable region comprises
a CDR1 comprising SEQ ID NO: 110;
a CDR2 comprising SEQ ID NO: 111; and
a CDR3 comprising SEQ ID NO: 112;
when determined according to Kabat numbering, optionally wherein:
the immunoglobulin heavy chain variable region comprises
a CDR1 comprising SEQ ID NO: 78,
a CDR2 comprising SEQ ID NO: 94; and
a CDR3 comprising SEQ ID NO: 98;
and the immunoglobulin light chain variable region comprises
a CDR1 comprising SEQ ID NO: 99,
a CDR2 comprising SEQ ID NO: 103; and
a CDR3 comprising SEQ ID NO: 105,
when determined according to Kabat numbering;
(b) the immunoglobulin heavy chain variable region comprises SEQ ID NO: 113 and the immunoglobulin light chain variable region comprises SEQ ID NO: 114;
(c) the immunoglobulin heavy chain variable region comprises at least 80% sequence identity to any one of SEQ ID NOs: 37-69, and/or comprises at least the CDR regions of any one of SEQ ID NOs: 37-69, and the immunoglobulin light chain variable region comprises at least 80% sequence identity to any one of SEQ ID NO: 70-77, and/or comprises at least the CDR regions of any one of SEQ ID NO: 70-77;
optionally wherein the immunoglobulin heavy chain variable region comprises at least 80% sequence identity to any one of SEQ ID NOs: 68 or 69, and/or comprises at least the CDR regions of any one of SEQ ID NOs: 68 or 69, and the immunoglobulin light chain variable region comprises at least 80% sequence identity to SEQ ID NO: 77, and/or comprises at least the CDR regions of SEQ ID NO: 77;
(d) the immunoglobulin heavy chain variable region comprises:
a CDR1 comprising SEQ ID NO: 146;
a CDR2 comprising SEQ ID NO: 147, and
a CDR3 comprising SEQ ID NO: 148;
and the immunoglobulin light chain variable region comprises
a CDR1 comprising SEQ ID NO: 138;
a CDR2 comprising SEQ ID NO: 139; and
a CDR3 comprising SEQ ID NO: 140 or 149;
when determined according to Kabat numbering; optionally wherein:
the immunoglobulin heavy chain variable region comprises:
a CDR1 comprising SEQ ID NO: 117 or 124;
a CDR2 comprising SEQ ID NO: 127; and
a CDR3 comprising SEQ ID NO: 136;
and the immunoglobulin light chain variable region comprises a CDR1 comprising SEQ ID NO: 138;
a CDR2 comprising SEQ ID NO: 139; and
a CDR3 comprising SEQ ID NO: 144 or 145;
when determined according to Kabat numbering;
(e) the immunoglobulin heavy chain variable region comprises SEQ ID NO: 150 and the immunoglobulin light chain variable region comprises SEQ ID NO: 151;
or
(f) the immunoglobulin heavy chain variable region comprises at least 80% sequence identity to any one of SEQ ID NOs: 1-24, and/or comprises at least the CDR regions of any one of SEQ ID NOs: 1-24, and the immunoglobulin light chain variable region comprises at least 80% sequence identity to SEQ ID NOs: 25-36, and/or comprises at least the CDR regions of any one of SEQ ID NOs: 25-36;
optionally wherein the immunoglobulin heavy chain variable region comprises at least 80% sequence identity to any one of SEQ ID NOs: 16, 23, or 24, and/or comprises at least the CDR regions of any one of SEQ ID NOs: 16, 23, or 24, and the immunoglobulin light chain variable region comprises at least 80% sequence identity to SEQ ID NO: 28 or 35, and/or comprises at least the CDR regions of SEQ ID NO: 28 or 35.

2.-33. (canceled)

34. The CD122 binding agent of claim 1, wherein the CD122 binding agent is an antibody, an antigen-binding antibody fragment, or a conjugate thereof.

35. The CD122 binding agent of claim 1, wherein the CD122 binding agent is a F(ab′)₂, Fab′, Fab, Fv, scFv, dsFv, or a single chain binding polypeptide.

36. The CD122 binding agent of claim 1, wherein the CD122 binding agent comprises an IgG Fc region that binds an Fc receptor on an antigen presenting cell, optionally an Fc region of IgG1.

37. A pharmaceutical composition comprising (a) the CD122 binding agent of claim 1, and (b) a pharmaceutically acceptable carrier.

38. A method of inhibiting an immune response in a mammal, which method comprises administering the CD122 binding agent of claim 1 to the mammal.

39. A method of treating an inflammatory or autoimmune disease in a mammal, which method comprises administering the CD122 binding agent of claim 1 to a mammal.

40. The method of claim 39, wherein the inflammatory or autoimmune disease is psoriasis, psoriatic arthritis, rheumatoid arthritis, contact hypersensitivity, allergic disease, eosinophilia, eosinophilic esophagitis, respiratory diseases such as asthma and ARDS (acute respiratory distress syndrome), Giant Cell Arteritis, Polymyalgia Rheumatica, Primary Sjögren's Syndrome, TNF-refractory Rheumatoid Arthritis, Alopecia Areata, Primary Biliary Cholangitis (PBC), Graft vs Host Disease (GvHD), Vitiligo, ANCA Vasculitis, Type 1 Diabetes, inflammatory bowel disease including Crohn's disease and ulcerative colitis, asthma, chronic periodontitis, recurrent fetal loss, celiac disease, transplant rejection or Noninfectious Uveitis.

41. The method of claim 39, further comprising administering a CD28 antagonist or JAK kinase inhibitor to the mammal.

42. A nucleic acid encoding the immunoglobulin heavy chain and/or immunoglobulin light chain of the CD122 binding agent of claim 1, optionally in a vector.

43. A cell that expresses the CD122 binding agent of claim 1.

44. A method of preparing the CD122 binding agent of claim 1, the method comprising expressing in a cell a nucleic acid sequence encoding the immunoglobulin heavy chain and a nucleic acid sequence encoding the immunoglobulin light chain of the CD122 binding agent.

45.-47. (canceled)


SEQUENCE LISTING (165 sequences)

SEQ ID NO: 1 - protein, 117 aa, synthetic construct
DVQLQQSGAELARPGASVKLSCTVSGFNFKDSLMHWVTQGPERGLRWIGWIDSENGETEYAPGFQDKATITADTSSSTVYLHLSSLTSEDTAIYYCARGGPWFLSYWGPGTTLTVSS

SEQ ID NO: 2 - protein, 117 aa, synthetic construct
EVQLVQSGAEVKKPGATVKISCKVSGFNFKDSLMHWVQQAPGKGLEWMGWIDSENGETEYAPGFQDRVTITADTSTDTAYMELSSLRSEDTAVYYCARGGPWFLSYWGQGTTVTVSS

SEQ ID NO: 3 - protein, 117 aa, synthetic construct
EVQLVQSGAEVKKPGATVKISCKVSGFNFKDSLMHWVQQAPGKGLEWMGWIDSETGETEYAPGFQDRVTITADTSTDTAYMELSSLRSEDTAVYYCARGGPWFLSYWGQGTTVTVSS

SEQ ID NO: 4 - protein, 117 aa, synthetic construct
EVQLVQSGAEVKKPGATVKISCKVSGFNFKDSLMHWVQQAPGKGLEWMGWIDSESGETEYAPGFQDRVTITADTSTDTAYMELSSLRSEDTAVYYCARGGPWFLSYWGQGTTVTVSS

SEQ ID NO: 5 - protein, 117 aa, synthetic construct
EVQLVQSGAEVKKPGATVKISCKVSGFNFKDSLMHWVQQAPGKGLEWMGWIDSEQGETEYAPGFQDRVTITADTSTDTAYMELSSLRSEDTAVYYCARGGPWFLSYWGQGTTVTVSS

SEQ ID NO: 6 - protein, 117 aa, synthetic construct
EVQLVQSGAEVKKPGATVKISCKVSGFNFKDSLMHWVQQAPGKGLEWMGWIDSEHGETEYAPGFQDRVTITADTSTDTAYMELSSLRSEDTAVYYCARGGPWFLSYWGQGTTVTVSS

SEQ ID NO: 7 - protein, 117 aa, synthetic construct
EVQLVQSGAEVKKPGATVKISCKVSGFNFKDSLMHWVQQAPGKGLEWMGWIDSEAGETEYAPGFQDRVTITADTSTDTAYMELSSLRSEDTAVYYCARGGPWFLSYWGQGTTVTVSS

SEQ ID NO: 8 - protein, 117 aa, synthetic construct
EVQLVQSGAEVKKPGATVKISCKVSGFNFKDSLVHWVQQAPGKGLEWMGWIDSENGETEYAPGFQDRVTITADTSTDTAYMELSSLRSEDTAVYYCARGGPWFLSYWGQGTTVTVSS

SEQ ID NO: 9 - protein, 117 aa, synthetic construct
EVQLVQSGAEVKKPGATVKISCKVSGFNFKDSLLHWVQQAPGKGLEWMGWIDSENGETEYAPGFQDRVTITADTSTDTAYMELSSLRSEDTAVYYCARGGPWFLSYWGQGTTVTVSS

SEQ ID NO: 10 - protein, 117 aa, synthetic construct
EVQLVQSGAEVKKPGATVKISCKVSGFNFKDSLIHWVQQAPGKGLEWMGWIDSENGETEYAPGFQDRVTITADTSTDTAYMELSSLRSEDTAVYYCARGGPWFLSYWGQGTTVTVSS

SEQ ID NO: 11 - protein, 117 aa, synthetic construct
EVQLVQSGAEVKKPGATVKISCKVSGFNFKDSLMHWVQQAPGKGLEWMGWIDNENGETEYAPGFQDRVTITADTSTDTAYMELSSLRSEDTAVYYCARGGPWFLSYWGQGTTVTVSS

SEQ ID NO: 12 - protein, 117 aa, synthetic construct
EVQLVQSGAEVKKPGATVKISCKVSGFNFKLSLMHWVQQAPGKGLEWMGWIDSENGETEYAPGFQDRVTITADTSTDTAYMELSSLRSEDTAVYYCARGGPWFLSYWGQGTTVTVSS

SEQ ID NO: 13 - protein, 117 aa, synthetic construct
EVQLVQSGAEVKKPGATVKISCKVSGFNFKYSLMHWVQQAPGKGLEWMGWIDSENGETEYAPGFQDRVTITADTSTDTAYMELSSLRSEDTAVYYCARGGPWFLSYWGQGTTVTVSS

SEQ ID NO: 14 - protein, 117 aa, synthetic construct
EVQLVQSGAEVKKPGATVKISCKVSGFNFKESLMHWVQQAPGKGLEWMGWIDSENGETEYAPGFQDRVTITADTSTDTAYMELSSLRSEDTAVYYCARGGPWFLSYWGQGTTVTVSS

SEQ ID NO: 15 - protein, 117 aa, synthetic construct
EVQLVQSGAEVKKPGATVKISCKVSGFNFKYSLMHWVQQAPGKGLEWMGWIDNEAGETEYAPGFQDRVTITADTSTDTAYMELSSLRSEDTAVYYCARGGPWFLSYWGQGTTVTVSS

SEQ ID NO: 16 - protein, 117 aa, synthetic construct
EVQLVQSGAEVKKPGATVKISCKVSGFNFKYSLIHWVQQAPGKGLEWMGWIDNEAGETEYAPGFQDRVTITADTSTDTAYMELSSLRSEDTAVYYCARGGPWFLSYWGQGTTVTVSS

SEQ ID NO: 17 - protein, 117 aa, synthetic construct
EVQLVQSGAEVKKPGATVKISCKVSGFNFKLSLMHWVQQAPGKGLEWMGWIDNEAGETEYAPGFQDRVTITADTSTDTAYMELSSLRSEDTAVYYCARGGPWFLSYWGQGTTVTVSS

SEQ ID NO: 18 - protein, 117 aa, synthetic construct
EVQLVQSGAEVKKPGATVKISCKVSGFNFKLSLIHWVQQAPGKGLEWMGWIDNEAGETEYAPGFQDRVTITADTSTDTAYMELSSLRSEDTAVYYCARGGPWFLSYWGQGTTVTVSS

SEQ ID NO: 19 - protein, 117 aa, synthetic construct
EVQLVQSGAEVKKPGATVKISCKVSGFNFKESLMHWVQQAPGKGLEWMGWIDVEAGETEYAPGFQDRVTITADTSTDTAYMELSSLRSEDTAVYYCARGGPWFLSYWGQGTTVTVSS

SEQ ID NO: 20 - protein, 117 aa, synthetic construct
EVQLVQSGAEVKKPGATVKISCKVSGFNFKESLMHWVQQAPGKGLEWMGWIDNEAGETEYAPGFQDRVTITADTSTDTAYMELSSLRSEDTAVYYCARGGPWFLSYWGQGTTVTVSS

SEQ ID NO: 21 - protein, 117 aa, synthetic construct
EVQLVQSGAEVKKPGATVKISCKVSGFNFKESLIHWVQQAPGKGLEWMGWIDVEAGETEYAPGFQDRVTITADTSTDTAYMELSSLRSEDTAVYYCARGGPWFLSYWGQGTTVTVSS

SEQ ID NO: 22 - protein, 117 aa, synthetic construct
EVQLVQSGAEVKKPGATVKISCKVSGFNFKESLIHWVQQAPGKGLEWMGWIDNEAGETEYAPGFQDRVTITADTSTDTAYMELSSLRSEDTAVYYCARSGPWFLSYWGQGTTVTVSS

SEQ ID NO: 23 - protein, 117 aa, synthetic construct
EVQLVQSGAEVKKPGATVKISCKVSGFNFKESLIHWVQQAPGKGLEWMGWIDNEAGETEYAPGFQDRVTITADTSTDTAYMELSSLRSEDTAVYYCARGGPWFLSYWGQGTTVTVSS

SEQ ID NO: 24 - protein, 117 aa, synthetic construct; identical to SEQ ID NO: 23

SEQ ID NO: 25 - protein, 108 aa, synthetic construct
DIVLTQTPATLSVTPGDSVSLSCRASQIISNNLHWYQQKSHESPRLLIIYGLQSISGIPYRFSGRRSGTDFTLNINSVEIEDFGMYFCQQSNNWPWTFGGGTKLDIRR

SEQ ID NO: 26 - protein, 108 aa, synthetic construct
EIVMTQSPATLSVSPGERATLSCRASQIISNNLHWYQQKPGQAPRLLIYYGLQSISGIPARFSGSGSGTEFTLTISSLQSEDFAVYYCQQSNNWPWTFGQGTKLEIKR

SEQ ID NO: 27 - protein, 108 aa, synthetic construct
EIVMTQSPATLSVSPGERATLSCRASQIISNNLHWYQQKPGQAPRLLIYYGLQSISGIPARFSGSRSGTEFTLTISSLQSEDFAVYYCQQSNNWPWTFGQGTKLEIKR

SEQ ID NO: 28 - protein, 108 aa, synthetic construct
EIVMTQSPATLSVSPGERATLSCRASQIISNNLHWYQQKPGQAPRLLIIYGLQSISGIPARFSGSRSGTEFTLTISSLQSEDFAVYYCQQSNNWPWTFGQGTKLEIKR

SEQ ID NO: 29 - protein, 108 aa, synthetic construct
EIVMTQSPATLSVSPGERATLSCRASQIISNNLHWYQQKPGQAPRLLIIYGLQSISGIPARFSGSGSGTEFTLTISSLQSEDFAVYFCQQSNNWPWTFGQGTKLEIKR

SEQ ID NO: 30 - protein, 108 aa, synthetic construct
EIVMTQSPATLSVSPGERATLSCRASQIISNNLHWYQQKPGQAPRLLIYYGLQSISGIPARFSGSRSGTEFTLTISSLQSEDFAVYFCQQSNNWPWTFGQGTKLEIKR

SEQ ID NO: 31 - protein, 108 aa, synthetic construct
EIVMTQSPATLSVSPGERATLSCRASQIISNNLHWYQQKPGQAPRLLIIYGLQSISGIPARFSGSRSGTEFTLTISSLQSEDFAVYFCQQSNNWPWTFGQGTKLEIKR

SEQ ID NO: 32 - protein, 108 aa, synthetic construct
EIVMTQSPATLSVSPGERATLSCRASQIISNNLHWYQQKPGQAPRLLIIYGLQSISGIPARFSGSRSGTEFTLTISSLQSEDFAVYFCQQSNNWPYTFGQGTKLEIKR

SEQ ID NO: 33 - protein, 108 aa, synthetic construct
EIVMTQSPATLSVSPGERATLSCRASQIISNNLHWYQQKPGQAPRLLIIYGLQSISGIPARFSGSRSGTEFTLTISSLQSEDFAVYFCQQSNNWPFTFGQGTKLEIKR

SEQ ID NO: 34 - protein, 108 aa, synthetic construct
EIVMTQSPATLSVSPGERATLSCRASQIISNNLHWYQQKPGQAPRLLIIYGLQSISGIPARFSGSRSGTEFTLTISSLQSEDFAVYFCQQSNNYPWTFGQGTKLEIKR

SEQ ID NO: 35 - protein, 108 aa, synthetic construct
EIVMTQSPATLSVSPGERATLSCRASQIISNNLHWYQQKPGQAPRLLIIYGLQSISGIPARFSGSRSGTEFTLTISSLQSEDFAVYFCQQSNNFPWTFGQGTKLEIKR

SEQ ID NO: 36 - protein, 108 aa, synthetic construct
EIVMTQSPATLSVSPGERATLSCRASQIISNNLHWYQQKPGQAPRLLIIYGLQSISGIPARFSGSRSGTEFTLTISSLQSEDFAVYFCQQSNNFPYTFGQGTKLEIKR

SEQ ID NO: 37 - protein, 116 aa, synthetic construct
QVQLQQSGAELMKPGASVKISCKSTGYTFSRYWIEWVKQRPGHGLEWIGEILPGSGSINHNEKFQGKATFTADTSSNTVYMQLSSLTSEDSAVYFCTRHYGSFDYWGQGTTLTVSS

SEQ ID NO: 38 - protein, 116 aa, synthetic construct
QVQLVQSGAEVKKPGASVKVSCKASGYTFSRYWIEWVRQAPGQGLEWMGEILPGSGSINHNEKFQGRVTMTRDTSTSTVYMELSSLRSEDTAVYYCTRHYGSFDYWGQGTTVTVSS

SEQ ID NO: 39 - protein, 116 aa, synthetic construct
QVQLVQSGAEVKKPGASVKVSCKASGYTFTRYWIEWVRQAPGQGLEWMGEILPGSGSINHNEKFQGRVTMTRDTSTSTVYMELSSLRSEDTAVYYCTRHYGSFDYWGQGTTVTVSS

SEQ ID NO: 40 - protein, 116 aa, synthetic construct
QVQLVQSGAEVKKPGASVKVSCKASGYTFSRYWIQWVRQAPGQGLEWMGEILPGSGSINHNEKFQGRVTMTRDTSTSTVYMELSSLRSEDTAVYYCTRHYGSFDYWGQGTTVTVSS

SEQ ID NO: 41 - protein, 116 aa, synthetic construct
QVQLVQSGAEVKKPGASVKVSCKASGYTFSRYWINWVRQAPGQGLEWMGEILPGSGSINHNEKFQGRVTMTRDTSTSTVYMELSSLRSEDTAVYYCTRHYGSFDYWGQGTTVTVSS

SEQ ID NO: 42 - protein, 116 aa, synthetic construct
QVQLVQSGAEVKKPGASVKVSCKASGYTFSRYWIEWVRQAPGQGLEWMGEILPGSGSIDHNEKFQGRVTMTRDTSTSTVYMELSSLRSEDTAVYYCTRHYGSFDYWGQGTTVTVSS

SEQ ID NO: 43 - protein, 116 aa, synthetic construct
QVQLVQSGAEVKKPGASVKVSCKASGYTFSRQWIEWVRQAPGQGLEWMGEILPGSGSINHNEKFQGRVTMTRDTSTSTVYMELSSLRSEDTAVYYCTRHYGSFDYWGQGTTVTVSS

SEQ ID NO: 44 - protein, 116 aa, synthetic construct
QVQLVQSGAEVKKPGASVKVSCKASGYTFSRYWIEWVRQAPGQGLEWMGEILPGSGSINHNEKFQGRVTFTADTSTSTVYMELSSLRSEDTAVYYCTRHYGSFDYWGQGTTVTVSS

SEQ ID NO: 45 - protein, 116 aa, synthetic construct
QVQLVQSGAEVKKPGASIKVSCKASGYTFSRYWIQWVRQAPGQGLEWMGEILPGSGSINHNEKFQGRVTMTRDTSTSTVYMELSSLRSEDTAVYYCTRHYGSFDYWGQGTTVTVSS

SEQ ID NO: 46 - protein, 116 aa, synthetic construct
QVQLVQSGAEVKKPGASVKVSCKVSGYTFSRYWIQWVRQAPGQGLEWMGEILPGSGSINHNEKFQGRVTMTRDTSTSTVYMELSSLRSEDTAVYYCTRHYGSFDYWGQGTTVTVSS

SEQ ID NO: 47 - protein, 116 aa, synthetic construct
QVQLVQSGAEVKKPGASVKVSCKASGYTFITYWIEWVRQAPGQGLEWMGEILPGSGSINHNEKFQGRVTMTRDTSTSTVYMELSSLRSEDTAVYYCTRHYGSFDYWGQGTTVTVSS

SEQ ID NO: 48 - protein, 116 aa, synthetic construct
QVQLVQSGAEVKKPGASVKVSCKASGYKFSRYWIQWVRQAPGQGLEWMGEILPGSGSINHNEKFQGRVTMTRDTSTSTVYMELSSLRSEDTAVYYCTRHYGSFDYWGQGTTVTVSS

SEQ ID NO: 49 - protein, 116 aa, synthetic construct
QVQLVQSGAEVKKPGASVKVSCKASGYLFSRYWIQWVRQAPGQGLEWMGEILPGSGSINHNEKFQGRVTMTRDTSTSTVYMELSSLRSEDTAVYYCTRHYGSFDYWGQGTTVTVSS

SEQ ID NO: 50 - protein, 116 aa, synthetic construct
QVQLVQSGAEVKKPGASVKVSCKASGYTFGRYWIQWVRQAPGQGLEWMGEILPGSGSINHNEKFQGRVTMTRDTSTSTVYMELSSLRSEDTAVYYCTRHYGSFDYWGQGTTVTVSS

SEQ ID NO: 51 - protein, 116 aa, synthetic construct
QVQLVQSGAEVKKPGASVKVSCKASGYTFISYWIQWVRQAPGQGLEWMGEILPGSGSINHNEKFQGRVTMTRDTSTSTVYMELSSLRSEDTAVYYCTRHYGSFDYWGQGTTVTVSS

SEQ ID NO: 52 - protein, 116 aa, synthetic construct
QVQLVQSGAEVKKPGASVKVSCKASGYTFSRYWIEWVRQAPGQGLEWMGEILPGSGSINHNEKFQGRVTFTADTSTSTVYMELSSLRSEDTAVYFCTRHYGSFDYWGQGTTVTVSS

SEQ ID NO: 53 - protein, 116 aa, synthetic construct
QVQLVQSGAEVKKPGASVKVSCKASGYIFSRYWIQWVRQAPGQGLEWMGEILPGSVSINHNEKFQGRVTMTRDTSTSTVYMELSSLRSEDTAVYYCTRHYGSFDYWGQGTTVTVSS

SEQ ID NO: 54 - protein, 116 aa, synthetic construct
QVQLVQSGAEVKKPGASVKVSCKASGYRFSTQWIEWVRQAPGQGLEWMGEILPGSGSINHNEKFQGRVTMTRDTSTSTVYMELSSLRSEDTAVYYCTRHYGSFDYWGQGTTVTVSS

SEQ ID NO: 55 - protein, 116 aa, synthetic construct
QVQLVQSGAEVKKPGASVKVSCKASGYTFSSHWIQWVRQAPGQGLEWMGEILPGSGSINHNEKFQGRVTMTRDTSTSTVYMELSSLRSEDTAVYYCTRHYGSFDYWGQGTTVTVSS

SEQ ID NO: 56 - protein, 116 aa, synthetic construct
QVQLVQSGAEVKKPGASVKVSCKASGYTFARHWIGWVRQAPGQGLEWMGEILPGSGSINHNEKFQGRVTMTRDTSTSTVYMELSSLRSEDTAVYYCTRHYGSFDYWGQGTTVTVSS

SEQ ID NO: 57 - protein, 116 aa, synthetic construct
QVQLVQSGAEVKKPGASVKVSCKASGYTFTSQWIEWVRQAPGQGLEWMGEILPGSGSINHNEKFQGRVTMTRDTSTSTVYMELSSLRSEDTAVYYCTRHYGSFDYWGQGTTVTVSS

SEQ ID NO: 58 - protein, 116 aa, synthetic construct
QVQLVQSGAEVKKPGASVKVSCKASGYKFSSYWIQWVRQAPGQGLEWMGEILPGSGSINHNEKFQGRVTMTRDTSTSTVYMELSSLRSEDTAVYYCTRHYGSFDYWGQGTTVTVSS

SEQ ID NO: 59 - protein, 116 aa, synthetic construct
QVQLVQSGAEVKKPGASVKVSCKASGYSFSKHWIQWVRQAPGQGLEWMGEILPGSGSINHNEKFQGRVTMTRDTSTSTVYMELSSLRSEDTAVYYCTRHYGSFDYWGQGTTVTVSS

SEQ ID NO: 60 - protein, 116 aa, synthetic construct
QVQLVQSGAEVKKPGASVKVSCKASGYPFTSYWIQWVRQAPGQGLEWMGEILPGSGSINHNEKFQGRVTMTRDTSTSTVYMELSSLRSEDTAVYYCTRHYGSFDYWGQGTTVTVSS

SEQ ID NO: 61 - protein, 116 aa, synthetic construct
QVQLVQSGAEVKKPGASVKVSCKASGYSFRRHWIQWVRQAPGQGLEWMGEILPGSGSINHNEKFQGRVTMTRDTSTSTVYMELSSLRSEDTAVYYCTRHYGSFDYWGQGTTVTVSS

SEQ ID NO: 62 - protein, 116 aa, synthetic construct
QVQLVQSGAEVKKPGASVKVSCKASGYTFSRYWIEWVRQAPGQGLEWMGEISPGVDTINHNEKFQGRVTMTRDTSTSTVYMELSSLRSEDTAVYYCTRHYGSFDYWGQGTTVTVSS

SEQ ID NO: 63 - protein, 116 aa, synthetic construct
QVQLVQSGAEVKKPGASIKVSCKASGYTFTRFWIQWVRQAPGQGLEWMGEILPGSGSINHNEKFQGRVTMTRDTSTSTVYMELSSLRSEDTAVYYCTRHYGSFDYWGQGTTVTVSS

SEQ ID NO: 64 - protein, 116 aa, synthetic construct
QVQLVQSGAEVKKPGASVKVSCKVSGYTFTRFWIQWVRQAPGQGLEWMGEILPGSGSINHNEKFQGRVTMTRDTSTSTVYMELSSLRSEDTAVYYCTRHYGSFDYWGQGTTVTVSS

SEQ ID NO: 65 - protein, 116 aa, synthetic construct
QVQLVQSGAEVKKPGASVKVSCKASGYIFASNWIQWVRQAPGQGLEWMGEILPGSGSINHNEKFQGRVTMTRDTSTSTVYMELSSLRSEDTAVYYCTRHYGSFDYWGQGTTVTVSS

SEQ ID NO: 66 - protein, 116 aa, synthetic construct
QVQLVQSGAEVKKPGASVKVSCKASGYSFIIHWIQWVRQAPGQGLEWMGEILPGSGSINHNEKFQGRVTMTRDTSTSTVYMELSSLRSEDTAVYYCTRHYGSFDYWGQGTTVTVSS

SEQ ID NO: 67 - protein, 116 aa, synthetic construct
QVQLVQSGAEVKKPGASVKVSCKASGYAFVDNWIQWVRQAPGQGLEWMGEILPGSGSINHNEKFQGRVTMTRDTSTSTVYMELSSLRSEDTAVYYCTRHYGSFDYWGQGTTVTVSS

SEQ ID NO: 68 - protein, 116 aa, synthetic construct
QVQLVQSGAEVKKPGASVKVSCKASGYTFTRFWIQWVRQAPGQGLEWMGEILPGSGSINHNEKFQGRVTMTRDTSTSTVYMELSSLRSEDTAVYYCTRHYGSFDYWGQGTTVTVSS

SEQ ID NO: 69 - protein, 116 aa, synthetic construct; identical to SEQ ID NO: 68

SEQ ID NO: 70 - protein, 108 aa, synthetic construct
EIVMTQSPKFMSTSVGDRVSVTCKASQNVGTDVAWYQQKPGQSPKALIYSASYRYNGVPDRFTGSGSGTDFTLTISNVQSEDLAEYFCHQYSSYPFTFGSGTKLEIKR

SEQ ID NO: 71 - protein, 108 aa, synthetic construct
DIQMTQSPSSLSASVGDRVTITCKASQNVGTDVAWFQQKPGKAPKSLIYSASYRYNGVPSRFSGSGSGTDFTLTISSLQPEDFATYYCHQYSSYPFTFGQGTKLEIKR

SEQ ID NO: 72 - protein, 108 aa, synthetic construct
DIQMTQSPSSLSASVGDRVTITCKASQNVGTDVAWYQQKPGKAPKSLIYSASYRYSGVPSRFSGSGSGTDFTLTISSLQPEDFATYFCHQYSSYPFTFGQGTKLEIKR

SEQ ID NO: 73 - protein, 108 aa, synthetic construct
DIQMTQSPSSLSASVGDRVTITCKASQNVDTDVAWYQQKPGKAPKSLIYSASYRYSGVPSRFSGSGSGTDFTLTISSLQPEDFATYFCHQYSSYPFTFGQGTKLEIKR

SEQ ID NO: 74 - protein, 108 aa, synthetic construct
DIQMTQSPSSLSASVGDRVTITCKASQNVGTNVAWYQQKPGKAPKSLIYSASYRYSGVPSRFSGSGSGTDFTLTISSLQPEDFATYFCHQYSSYPFTFGQGTKLEIKR

SEQ ID NO: 75 - protein, 108 aa, synthetic construct
DIQMTQSPSSLSASVGDRVTITCKASQNVGTEVAWYQQKPGKAPKSLIYSASYRYSGVPSRFSGSGSGTDFTLTISSLQPEDFATYFCHQYSSYPFTFGQGTKLEIKR

SEQ ID NO: 76 - protein, 108 aa, synthetic construct
DIQMTQSPSSLSASVGDRVTITCKASQNVGTDVAWYQQKPGKAPKSLIYSASYRYSGVPSRFSGSGSGTDFTLTISSLQPEDFATYFCQQYSSYPFTFGQGTKLEIKR

SEQ ID NO: 77 - protein, 108 aa, synthetic construct
DIQMTQSPSSLSASVGDRVTITCKASQNVGTDVAWYQQKPGKAPKSLIYSASYRYSGVPSRFSGSGSGTDFTLTISSLQPEDFATYFCHQYESYPFTFGQGTKLEIKR

SEQ ID NO: 78 - protein, 5 aa, synthetic construct
RFWIQ

SEQ ID NO: 79 - protein, 5 aa, synthetic construct
RYWIE

SEQ ID NO: 80 - protein, 5 aa, synthetic construct
RYWIQ

SEQ ID NO: 81 - protein, 5 aa, synthetic construct
RYWIN

SEQ ID NO: 82 - protein, 5 aa, synthetic construct
RQWIE

SEQ ID NO: 83 - protein, 5 aa, synthetic construct
TYWIE

SEQ ID NO: 84 - protein, 5 aa, synthetic construct
SYWIQ

SEQ ID NO: 85 - protein, 5 aa, synthetic construct
TQWIE

SEQ ID NO: 86 - protein, 5 aa, synthetic construct
SHWIQ

SEQ ID NO: 87 - protein, 5 aa, synthetic construct
RHWIG

SEQ ID NO: 88 - protein, 5 aa, synthetic construct
SQWIE

SEQ ID NO: 89 - protein, 5 aa, synthetic construct
KHWIQ

SEQ ID NO: 90 - protein, 5 aa, synthetic construct
RHWIQ

SEQ ID NO: 91 - protein, 5 aa, synthetic construct
SNWIQ

SEQ ID NO: 92 - protein, 5 aa, synthetic construct
IHWIQ

SEQ ID NO: 93 - protein, 5 aa, synthetic construct
DNWIQ

SEQ ID NO: 94 - protein, 17 aa, synthetic construct
EILPGSGSINHNEKFQG

SEQ ID NO: 95 - protein, 17 aa, synthetic construct
EILPGSGSIDHNEKFQG

SEQ ID NO: 96 - protein, 17 aa, synthetic construct
EILPGSVSINHNEKFQG

SEQ ID NO: 97 - protein, 17 aa, synthetic construct
EISPGVDTINHNEKFQG

SEQ ID NO: 98 - protein, 7 aa, synthetic construct
HYGSFDY

SEQ ID NO: 99 - protein, 11 aa, synthetic construct
KASQNVGTDVA

SEQ ID NO: 100 - protein, 11 aa, synthetic construct
KASQNVDTDVA

SEQ ID NO: 101 - protein, 11 aa, synthetic construct
KASQNVGTNVA

SEQ ID NO: 102 - protein, 11 aa, synthetic construct
KASQNVGTEVA

SEQ ID NO: 103 - protein, 7 aa, synthetic construct
SASYRYS

SEQ ID NO: 104 - protein, 7 aa, synthetic construct
SASYRYN

SEQ ID NO: 105 - protein, 10 aa, synthetic construct
HQYESYPFTF

SEQ ID NO: 106 - protein, 10 aa, synthetic construct
QQYSSYPFTF

SEQ ID NO: 107 - protein, 10 aa, synthetic construct
HQYSSYPFTF

SEQ ID NO: 108 - protein, 5 aa, synthetic construct
  VARIANT 1: note = R can be replaced by T, S, K, I or D
  VARIANT 2: note = Y can be replaced by Q, H, N or F
  VARIANT 5: note = Can be replaced by Q,N or G
RYWIE

SEQ ID NO: 109 - protein, 17 aa, synthetic construct
  VARIANT 3: note = L or S
  VARIANT 6: note = S or V
  VARIANT 7: note = G,V or D
  VARIANT 8: note = S or T
  VARIANT 10: note = N or D
EIXPGXXXIXHNEKFQG

SEQ ID NO: 110 - protein, 11 aa, synthetic construct
  VARIANT 7: note = G,D
  VARIANT 9: note = D,N,E
KASQNVXTXVA

SEQ ID NO: 111 - protein, 7 aa, synthetic construct
  VARIANT 7: note = N,S
SASYRYX

SEQ ID NO: 112 - protein, 10 aa, synthetic construct
  VARIANT 1: note = H or Q
  VARIANT 4: note = S or E
XQYXSYPFTF

SEQ ID NO: 113 - protein, 116 aa, synthetic construct
  VARIANT 5: note = Q or V
  VARIANT 11: note = L or V
  VARIANT 12: note = M or K
  VARIANT 18: note = V or I
  VARIANT 20: note = I or V
  VARIANT 24: note = S,A or V
  VARIANT 25: note = T or S
  VARIANT 28: note = T,K,L,I,R,S,P or A
  VARIANT 30: note = S,I,G,A,T,R or V
  VARIANT 31: note = R,Q,T,S,K or D
  VARIANT 32: note = Y,Q,H,N,F or S
  VARIANT 33: note = W or Q
  VARIANT 34: note = E,Q,N or G
  VARIANT 37: note = K or R
  VARIANT 39: note = R,A or T
  VARIANT 42: note = H or Q
  VARIANT 47: note = I or M
  VARIANT 51: note = L or S
  VARIANT 54: note = S or V
  VARIANT 55: note = G,V or D
  VARIANT 56: note = S or T
  VARIANT 58: note = N or D
  VARIANT 66: note = K or R
  VARIANT 67: note = A or V
  VARIANT 69: note = F or M
  VARIANT 70: note = A or R
  VARIANT 74: note = S or T
  VARIANT 75: note = N or S
  VARIANT 80: note = Q or E
  VARIANT 85: note = T or R
  VARIANT 89: note = S or T
  VARIANT 93: note = F or Y
  VARIANT 110: note = L or V
QVQLXQSGAEXXKPGASXKXSCKXXGYXFXXXXIXWVXQXPGXGLEWXGEIXPGXXXIXHNEKFQGXXTXTXDTSXXTVYMXLSSLXSEDXAVYXCTRHYGSFDYWGQGTTXTVSS

SEQ ID NO: 114 - protein, 109 aa, synthetic construct
  VARIANT 1: note = E or D
  VARIANT 3: note = V or Q
  VARIANT 9: note = K or S
  VARIANT 10: note = F or S
  VARIANT 11: note = M or L
  VARIANT 13: note = T or A
  VARIANT 20: note = S or T
  VARIANT 21: note = V or I
  VARIANT 30: note = G or D
  VARIANT 32: note = D,N or E
  VARIANT 36: note = Y or F
  VARIANT 42: note = Q or K
  VARIANT 43: note = Q or A
  VARIANT 46: note = A or S
  VARIANT 55: note = Y or S
  VARIANT 60: note = D or S
  VARIANT 63: note = T or S
  VARIANT 77: note = N or S
  VARIANT 78: note = V or L
  VARIANT 80: note = S or P
  VARIANT 83: note = L or F
  VARIANT 85: note = E or T
  VARIANT 87: note = F or Y
  VARIANT 89: note = H or Q
  VARIANT 92: note = S or E
  VARIANT 101: note = S or Q
XIXMTQSPXXXSXSVGDRVXXTCKASQNVXTXVAWXQQKPGXXPKXLIYSASYRXNGVPXRFXGSGSGTDFTLTISXXQXEDXAXYXCXQYXSYPFTFGSXGTKLEIKR

SEQ ID NO: 115 - protein, 446 aa, synthetic construct
QVQLVQSGAEVKKPGASVKVSCKASGYTFTRFWIQWVRQAPGQGLEWMGEILPGSGSINHNEKFQGRVTMTRDTSTSTVYMELSSLRSEDTAVYYCTRHYGSFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 214 aa, synthetic construct
DIQMTQSPSSLSASVGDRVTITCKASQNVGTDVAWYQQKPGKAPKSLIYSASYRYSGVPSRFSGSGSGTDFTLTISSLQPEDFATYFCHQYESYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 5 aa, synthetic construct
ESLIH

SEQ ID NO: 118 - protein, 5 aa, synthetic construct
DSLVH

SEQ ID NO: 119 - protein, 5 aa, synthetic construct
DSLLH

SEQ ID NO: 120 - protein, 5 aa, synthetic construct
DSLIH

SEQ ID NO: 121 - protein, 5 aa, synthetic construct
LSLMH

SEQ ID NO: 122 - protein, 5 aa, synthetic construct
YSLMH

SEQ ID NO: 123 - protein, 5 aa, synthetic construct
ESLMH

SEQ ID NO: 124 - protein, 5 aa, synthetic construct
YSLIH

SEQ ID NO: 125 - protein, 5 aa, synthetic construct
LSLIH

SEQ ID NO: 126 - protein, 5 aa, synthetic construct
DSLMH

SEQ ID NO: 127 - protein, 17 aa, synthetic construct
WIDNEAGETEYAPGFQD

SEQ ID NO: 128 - protein, 17 aa, synthetic construct
WIDNENGETEYAPGFQD

SEQ ID NO: 129 - protein, 17 aa, synthetic construct
WIDSENGETEYAPGFQD

SEQ ID NO: 130 - protein, 17 aa, synthetic construct
WIDVEAGETEYAPGFQD

SEQ ID NO: 131 - protein, 17 aa, synthetic construct
WIDSESGETEYAPGFQD

SEQ ID NO: 132 - protein, 17 aa, synthetic construct
WIDSETGETEYAPGFQD

SEQ ID NO: 133 - protein, 17 aa, synthetic construct
WIDSEQGETEYAPGFQD

SEQ ID NO: 134 - protein, 17 aa, synthetic construct
WIDSEHGETEYAPGFQD

SEQ ID NO: 135 - protein, 17 aa, synthetic construct
WIDSEAGETEYAPGFQD

SEQ ID NO: 136 - protein, 8 aa, synthetic construct
GGPWFLSY

SEQ ID NO: 137 - protein, 8 aa, synthetic construct
SGPWFLSY

SEQ ID NO: 138 - protein, 11 aa, synthetic construct
RASQIISNNLH

SEQ ID NO: 139 - protein, 7 aa, synthetic construct
YGLQSIS

SEQ ID NO: 140 - protein, 9 aa, synthetic construct
QQSNNFPYT

SEQ ID NO: 141 - protein, 9 aa, synthetic construct
QQSNNWPYT

SEQ ID NO: 142 - protein, 9 aa, synthetic construct
QQSNNWPFT

SEQ ID NO: 143 - protein, 9 aa, synthetic construct
QQSNNYPWT

SEQ ID NO: 144 - protein, 9 aa, synthetic construct
QQSNNFPWT

SEQ ID NO: 145 - protein, 9 aa, synthetic construct
QQSNNWPWT

SEQ ID NO: 146 - protein, 5 aa, synthetic construct
  VARIANT 1: note = D,L,Y or E
  VARIANT 4: note = M,V,L or I
DSLMH

SEQ ID NO: 147 - protein, 17 aa, synthetic construct
  VARIANT 4: note = S,N or V
  VARIANT 7: note = N,T,S,Q,H or A
WIDXEXGETEYAPGFQD

SEQ ID NO: 148 - protein, 8 aa, synthetic construct
  VARIANT 1: note = G or S
XGPWFLSY

SEQ ID NO: 149 - protein, 9 aa, synthetic construct
  VARIANT 6: note = W,Y or F
  VARIANT 8: note = W,Y or F
QQSNNXPXT

SEQ ID NO: 150 - protein, 118 aa, synthetic construct
  VARIANT 1: note = D or E
  VARIANT 5: note = L or V
  VARIANT 12: note = L or V
  VARIANT 13: note = A or K
  VARIANT 14: note = R or K
  VARIANT 18: note = S or T
  VARIANT 21: note = L or I
  VARIANT 24: note = T or K
  VARIANT 32: note = D,L,Y or E
  VARIANT 35: note = V,L or I
  VARIANT 39: note = T or Q
  VARIANT 41: note = G or A
  VARIANT 43: note = E or G
  VARIANT 44: note = R or K
  VARIANT 47: note = R or E
  VARIANT 49: note = I or M
  VARIANT 54: note = S,N or V
  VARIANT 56: note = N,T,S,Q,H or A
  VARIANT 68: note = K or R
  VARIANT 69: note = A or V
  VARIANT 77: note = S or T
  VARIANT 78: note = S or D
  VARIANT 80: note = V or A
  VARIANT 82: note = L or M
  VARIANT 83: note = H or E
  VARIANT 88: note = T or R
  VARIANT 94: note = I or V
  VARIANT 100: note = G or S
  VARIANT 110: note = P or Q
  VARIANT 114: note = L or V
XVQQXQQSGAEXXXPGAXVKXSCXVSGFNFKXSLXHWVXQXPXXGLXWXGWIDXEXGETEYAPGFQDXXTITADTSXXTXYXXLSSLXSEDTAXYYCARXGPWFLSYWGXGTTXTVSS

SEQ ID NO: 151 - protein, 108 aa, synthetic construct
  VARIANT 1: note = D or E
  VARIANT 4: note = L or M
  VARIANT 7: note = T or S
  VARIANT 14: note = T or S
  VARIANT 17: note = D or E
  VARIANT 18: note = S or R
  VARIANT 19: note = V or A
  VARIANT 20: note = S or T
  VARIANT 40: note = S or P
  VARIANT 41: note = H or G
  VARIANT 42: note = E or Q
  VARIANT 43: note = S or A
  VARIANT 49: note = I or Y
  VARIANT 60: note = Y or A
  VARIANT 65: note = R or S
  VARIANT 66: note = R or G
  VARIANT 70: note = D or E
  VARIANT 74: note = N or T
  VARIANT 76: note = N or S
  VARIANT 78: note = V or L
  VARIANT 79: note = E or Q
  VARIANT 80: note = I or S
  VARIANT 84: note = G or A
  VARIANT 85: note = M or V
  VARIANT 87: note = F or Y
  VARIANT 94: note = W,Y or F
  VARIANT 96: note = W,Y or F
  VARIANT 100: note = G or Q
  VARIANT 105: note = D or E
  VARIANT 107: note = R or K
XIVXTQXPATLSVXPGXXXXLSCRASQIISNNLHWYQQKXXXXPRLLIXYGLQSISGIPXRFSGXXSGTXFTLXIXSXXXEDFXXYXCQQSNNXPXTFGXGTKLXIXR

SEQ ID NO: 152 - protein, 447 aa, synthetic construct
EVQLVQSGAEVKKPGATVKISCKVSGFNFKESLIHWVQQAPGKGLEWMGWIDNEAGETEYAPGFQDRVTITADTSTDTAYMELSSLRSEDTAVYYCARGGPWFLSYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 214 aa, synthetic construct
EIVMTQSPATLSVSPGERATLSCRASQIISNNLHWYQQKPGQAPRLLIIYGLQSISGIPARFSGSRSGTEFTLTISSLQSEDFAVYFCQQSNNFPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 154 - DNA, 1341 bp, synthetic construct
  source 1..1341: mol_type = other DNA
gaggtccagctggtacagtctggggctgaggtgaagaagcctggggctacagtgaaaatctcctgcaaggtttctggcttcaactttaaagagagtcttattcactgggtgcaacaggcccctggaaaagggcttgagtggatgggatggattgataacgaagccggtgaaactgaatatgccccggggttccaggacagagtcaccataaccgcggacacgtctacagacacagcctacatggagctgagcagcctgagatctgaggacacggccgtgtattactgtgctcgaggggggccatggtttctaagttactgggggcaagggaccacggtcaccgtctcctcagcctccaccaagggcccatcggtcttcccgctagcaccctcctccaagagcacctctgggggcacagcggccctgggctgcctggtcaaggactacttccccgaacctgtgacggtgtcgtggaactcaggcgccctgaccagcggcgtgcacaccttcccggctgtcctacagtcctcaggactctactccctcagcagcgtggtgacagtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaggaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 155 - DNA, 642 bp, synthetic construct
  source 1..642: mol_type = other DNA
gaaatagtgatgacgcagtctccagccaccctgtctgtgtctccaggggaaagagccaccctctcctgcagggccagccaaattattagcaacaacctacactggtaccagcagaaacctggccaggctcccaggctcctcatcatatatgggctccagtccatctctggtatcccagccaggttcagtggcagtcgatctgggacagagttcactctcaccatcagcagcctgcagtctgaagattttgcagtttatttctgtcaacagagtaacaactttccgtggacgtttggccaggggaccaagctggagatcaaacggactgtggctgcaccatctgtcttcatcttcccgccatctgatgagcaattgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctcagctcgcccgtcacaaagagcttcaacaggggagagtgt

SEQ ID NO: 156 - DNA, 1332 bp, synthetic construct
  source 1..1332: mol_type = other DNA
gaggtccagctggtacagtctggggctgaggtgaagaagcctggggctacagtgaaaatctcctgcaaggtttctggcttcaactttaaagagagtcttattcactgggtgcaacaggcccctggaaaagggcttgagtggatgggatggattgataacgaagccggtgaaactgaatatgccccggggttccaggacagagtcaccataaccgcggacacgtctacagacacagcctacatggagctgagcagcctgagatctgaggacacggccgtgtattactgtgctcgaggggggccatggtttctaagttactgggggcaagggaccacggtcaccgtctcctcagcctccaccaagggcccatcggtcttcccgctagcaccctgctccaggagcacctccgagagcacagccgccctgggctgcctggtcaaggactacttccccgaaccagtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 157 - protein, 444 aa, synthetic construct
EVQLVQSGAEVKKPGATVKISCKVSGFNFKESLIHWVQQAPGKGLEWMGWIDNEAGETEYAPGFQDRVTITADTSTDTAYMELSSLRSEDTAVYYCARGGPWFLSYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 158 - DNA, 1332 bp, synthetic construct
  source 1..1332: mol_type = other DNA
gaggtccagctggtacagtctggggctgaggtgaagaagcctggggctacagtgaaaatctcctgcaaggtttctggcttcaactttaaatacagtcttattcactgggtgcaacaggcccctggaaaagggcttgagtggatgggatggattgataacgaagccggtgaaactgaatatgccccggggttccaggacagagtcaccataaccgcggacacgtctacagacacagcctacatggagctgagcagcctgagatctgaggacacggccgtgtattactgtgctcgaggggggccatggtttctaagttactgggggcaagggaccacggtcaccgtctcctcagcctccaccaagggcccatcggtcttcccgctagcaccctgctccaggagcacctccgagagcacagccgccctgggctgcctggtcaaggactacttccccgaaccagtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 159 - protein, 444 aa, synthetic construct
EVQLVQSGAEVKKPGATVKISCKVSGFNFKYSLIHWVQQAPGKGLEWMGWIDNEAGETEYAPGFQDRVTITADTSTDTAYMELSSLRSEDTAVYYCARGGPWFLSYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 160 - DNA, 642 bp, synthetic construct
  source 1..642: mol_type = other DNA
gaaatagtgatgacgcagtctccagccaccctgtctgtgtctccaggggaaagagccaccctctcctgcagggccagccaaattattagcaacaacctacactggtaccagcagaaacctggccaggctcccaggctcctcatcatatatgggctccagtccatctctggtatcccagccaggttcagtggcagtcgatctgggacagagttcactctcaccatcagcagcctgcagtctgaagattttgcagtttattattgtcaacagagtaacaactggccgtggacgtttggccaggggaccaagctggagatcaaacggactgtggctgcaccatctgtcttcatcttcccgccatctgatgagcaattgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctcagctcgcccgtcacaaagagcttcaacaggggagagtgt

SEQ ID NO: 161 - protein, 214 aa, synthetic construct
EIVMTQSPATLSVSPGERATLSCRASQIISNNLHWYQQKPGQAPRLLIIYGLQSISGIPARFSGSRSGTEFTLTISSLQSEDFAVYYCQQSNNWPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 162 - DNA, 1338 bp, synthetic construct
  source 1..1338: mol_type = other DNA
caggtgcagctggtgcagtctggggctgaggtgaagaagcctggggcctcagtgaaggtttcctgcaaggcatctggctacacattcaccaggttctggatacagtgggtgcgacaggcccctggacaagggcttgagtggatgggagagattttacctggaagtggtagtattaaccacaatgagaagttccagggcagagtcaccatgaccagggacacgtccacgagcacagtctacatggagctgagcagcctgagatctgaggacacggccgtgtattactgtacaagacactacggtagctttgactactgggggcaagggaccacggtcaccgtctcctcagcctccaccaagggcccatcggtcttcccgctagcaccctcctccaagagcacctctgggggcacagcggccctgggctgcctggtcaaggactacttccccgaacctgtgacggtgtcgtggaactcaggcgccctgaccagcggcgtgcacaccttcccggctgtcctacagtcctcaggactctactccctcagcagcgtggtgacagtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaggaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 163 - DNA, 642 bp, synthetic construct
  source 1..642: mol_type = other DNA
gacatccagatgacccagtctccatcctcactgtctgcatctgtaggagacagagtcaccatcacttgtaaggccagtcagaatgtgggtactgatgtagcctggtatcagcagaaaccagggaaagcccctaagtccctgatctattcggcatcctaccggtacagtggggtcccatcaaggttcagcggcagtggatctgggacagatttcactctcaccatcagcagcctgcagcctgaagattttgcaacttatttctgccaccaatatgagagttatccattcacgtttggccaggggaccaagctggagatcaaacggactgtggctgcaccatctgtcttcatcttcccgccatctgatgagcaattgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctcagctcgcccgtcacaaagagcttcaacaggggagagtgt

SEQ ID NO: 164 - DNA, 1329 bp, synthetic construct
  source 1..1329: mol_type = other DNA
caggtgcagctggtgcagtctggggctgaggtgaagaagcctggggcctcagtgaaggtttcctgcaaggcatctggctacacattcaccaggttctggatacagtgggtgcgacaggcccctggacaagggcttgagtggatgggagagattttacctggaagtggtagtattaaccacaatgagaagttccagggcagagtcaccatgaccagggacacgtccacgagcacagtctacatggagctgagcagcctgagatctgaggacacggccgtgtattactgtacaagacactacggtagctttgactactgggggcaagggaccacggtcaccgtctcctcagcctccaccaagggcccatcggtcttcccgctagcaccctgctccaggagcacctccgagagcacagccgccctgggctgcctggtcaaggactacttccccgaaccagtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 165 - protein, 443 aa, synthetic construct
QVQLVQSGAEVKKPGASVKVSCKASGYTFTRFWIQWVRQAPGQGLEWMGEILPGSGSINHNEKFQGRVTMTRDTSTSTVYMELSSLRSEDTAVYYCTRHYGSFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK